(19) United States
(12) Patent Application Publication
(10) Pub. No.: US 2014/0221287 A1
(43) Pub. Date: Aug. 7, 2014

(54) Amylin Peptides and Derivatives and Uses Thereof

(71) Applicants: Chengzao Sun, San Diego, CA (US); Manoj P. Samant, San Diego, CA (US); Swetha Neravetla, San Diego, CA (US)

(72) Inventors: Chengzao Sun, San Diego, CA (US); Manoj P. Samant, San Diego, CA (US); Swetha Neravetla, San Diego, CA (US)

(73) Assignees: ASTRAZENECA PHARMACEUTICALS LP, WILMINGTON, DE (US); AMYLIN PHARMACEUTICALS, LLC, SAN DIEGO, CA (US)

(21) Appl. No.: 14/119,574

(22) PCT Filed: May 24, 2012
(86) PCT No.: PCT/US12/39431

§ 371 Date: Nov. 22, 2013

Related U.S. Application Data
(60) Provisional application No. 61/489,781, filed on May 25, 2011.

(51) Int. Cl. C07K 14/575

(52) U.S. Cl. CPC C07K 14/575
USPC 514/6.7; 530/324; 514/9.7; 514/7.3; 514/6.9

(57) ABSTRACT

There are provided polypeptide conjugates having enhanced duration of biological activity, and methods of use thereof. The polypeptide conjugates include duration enhancing moieties, including water soluble polymers, bound to the polypeptide components of defined sequence. Methods of use are provided for treatment of metabolic disorders. Methods of use are provided for treatment of an eating disorder, insulin resistance, obesity, overweight, abnormal postprandial hyperglycemia, Type I diabetes, Type II diabetes, gestational diabetes, metabolic syndrome, dumping syndrome, hypertension, dyslipidemia, cardiovascular disease, hyper lipidemia, sleep apnea, cancer, pulmonary hypertension, cholescystitis, osteoarthritis, or short bowel syndrome.

### SEQUENCE LISTING

The instant application contains a Sequence Listing which has been submitted in ASCII format via EFS-Web and is hereby incorporated by reference in its entirety. Said ASCII copy, created on May 18, 2012, is named 123USPRO.txt and is 28,832 bytes in size.

### BACKGROUND OF THE INVENTION

There are provided polypeptide conjugates having enhanced duration of biological activity, and methods of use thereof. The polypeptide conjugates include a polypeptide component bound to one or more duration enhancing moieties, optionally through a linker. The polypeptide components included within the polypeptide conjugates are related to amylin, calcitonin and chimera thereof. The polypeptide conjugates further include duration enhancing moieties, including but not limited to water soluble polymers, bound to the polypeptide component of the peptide conjugate, optionally through a linker. The methods include treatment of obesity, diabetes, and other metabolic disorders.

Diabetes mellitus is a serious metabolic disease that is defined by the presence of chronically elevated levels of blood glucose (hyperglycemia). This state of hyperglycemia is the result of a relative or absolute lack of activity of the peptide hormone, insulin. Insulin is produced and secreted by the β-cells of the pancreas. Insulin is reported to promote glucose utilization, protein synthesis, and the formation and storage of neutral lipids. Glucose, the principal source of carbohydrate energy, is stored in the body as glycogen, a form of polymerized glucose, which may be converted back into glucose to meet metabolism requirements. Under normal conditions, insulin is secreted at both a basal rate and at enhanced rates following glucose stimulation, all to maintain metabolic homeostasis by the conversion of glucose into glycogen.

The term diabetes mellitus encompasses several different hyperglycemic states. These states include Type 1 (insulin-dependent diabetes mellitus or IDDM) and Type 2 (non-insulin-dependent diabetes mellitus or NIDDM) diabetes. The hyperglycemia present in individuals with Type I diabetes is associated with deficient, reduced, or nonexistent levels of insulin which are insufficient to maintain blood glucose levels within the physiological range. Treatment of Type 1 diabetes involves administration of replacement doses of insulin, generally by the parenteral route. The hyperglycemia present in individuals with Type II diabetes is initially associated with normal or elevated levels of insulin; however, these individuals are unable to maintain metabolic homeostasis due to a state of insulin resistance in peripheral tissues and liver and, as the disease advances, due to a progressive deterioration of the pancreatic β-cells which are responsible for the secretion of insulin. Thus, initial therapy of Type 2 diabetes may be based on diet and lifestyle changes augmented by therapy with oral hypoglycemic agents such as sulfonylureas. Insulin therapy is often required, however, especially in the latter stages of the disease, in attempting to produce some control of hyperglycemia and minimize complications of the disease. Thus, many Type 2 diabetics ultimately require insulin in order to survive.

Obesity and its associated disorders are common and very serious public health problems in the United States and throughout the world. Upper body obesity is the strongest risk factor known for type 2 diabetes mellitus and is a strong risk factor for cardiovascular disease. Obesity is a recognized risk factor for hypertension, atherosclerosis, congestive heart failure, stroke, gallbladder disease, osteoarthritis, sleep apnea, reproductive disorders such as polycystic ovarian syndrome, cancers of the breast, prostate, and colon, and increased incidence of complications of general anesthesia (see, e.g., Kopelman, Nature 404: 635-43 (2000)).

Obesity reduces life-span and carries a serious risk of the co-morbidities listed above, as well disorders such as infections, varicose veins, acanthosis nigricans, eczema, exercise intolerance, insulin resistance, hypertension hypercholesterolemia, cholelithiasis, orthopedic injury, and thromboembolic disease (Rissanen et al., Br. Med. J. 301: 835-7 (1990)). Obesity is also a risk factor for the group of conditions called insulin resistance syndrome, or “Syndrome X” and metabolic syndrome. The worldwide medical cost of obesity and associated disorders is enormous.

Amylin, as known in the art, is a peptide hormone synthesized by pancreatic β-cells that is co-secreted with insulin in response to nutrient intake. Thus, amylin has a metabolic function. The sequence of amylin is highly preserved across mammalian species and has structural similarities to calcitonin gene-related peptide (CGRP), the calcitonins, the intermedins, and adrenomedullin. See Young A., 2005, Amylin: Physiology and Pharmacology. In: August J T, Murad F, Granner, D (eds), AMYLIN: PHYSIOLOGY AND PHARMACOLOGY, Elsevier Academic Press: San Diego, Calif., USA, pp 1-18. The glucoregulatory actions of amylin complement those of insulin by regulating the rate of glucose appearance in the circulation via suppression of nutrient-stimulated glucagon secretion and slowing gastric emptying. See Young, 1997, Curr Opin Endocrinol Diabetes 4:282-290. For example, in insulin-treated patients with diabetes, pramlintide, a synthetic analogue of human amylin, reduces postprandial glucose excursions by suppressing inappropriately elevated postprandial glucagon secretion and slowing gastric emptying. See e.g., Janes et al., 1996, Diabetes, 45(suppl 2):865 (abstract); Young et al., 1996, Drug Dev Res 37:231-248; Weyer et al., 2001, Curr Pharm Des 7:1353-1373; Hoogwerf et al., 2008, Vasc Health Risk Manag 4:355-362; Edelman et al., 2008, Biodrugs 22:375-386.

There are provided herein polypeptides conjugated with water-soluble polymers which provide enhanced duration of action.

All references cited herein are incorporated by reference in their entirety and for all purposes.

### BRIEF SUMMARY OF THE INVENTION

In summary, there are provided polypeptide conjugates having enhanced duration of biological activity, and methods of use thereof. The polypeptide components included within the polypeptide conjugates are related to amylin, calcitonin and chimera thereof. The polypeptide conjugates further include duration enhancing moieties, including but not limited to water soluble polymers, bound to the polypeptide components of the peptide conjugates, optionally through linkers. The methods include treatment of obesity, diabetes, and other metabolic disorders.

In a first aspect, there is provided a polypeptide conjugate which includes a polypeptide component and a duration enhancing moiety covalently linked thereto. The polypeptide component includes an amino acid sequence of residues 1-37 of Formula (I) following, wherein up to 25% of the amino acids set forth in Formula (I) may be deleted or substituted with a different amino acid:

(I)
(SEQ ID NO: 4)
X′-Xaa¹-Cys²-Asn³-Thr⁴-Ala⁵-Thr⁶-Cys⁷-Ala⁸-Thr⁹-
Gln¹⁰-Arg¹¹-Leu¹²-Ala¹³-Asn¹⁴-Phe¹⁵-Leu¹⁶-Val¹⁷-Xaa¹⁸-
Ser¹⁹-Ser²⁰-Xaa²¹-Asn²²-Phe²³-Xaa²⁴-Xaa²⁵-Xaa²⁶-Xaa²⁷-
Xaa²⁸-Xaa²⁹-Thr³⁰-Xaa³¹-Val³²-Gly³³-Xaa³⁴-Xaa³⁵-Thr³⁶-
Tyr³⁷-X.

Regarding Formula (I), X′ is hydrogen, an N-terminal capping group, a bond to a duration enhancing moiety, or a linker to a duration enhancing moiety, Xaa¹ is Lys or a bond, Xaa¹⁸ is Lys, Cys, or His, Xaa²¹ is Lys, Cys, or Asn, Xaa²⁴ is Lys, Cys, or Gly, Xaa²⁵ is Lys, Cys, or Pro, Xaa²⁶ is Lys, Cys, or Ile, Xaa²⁷ is Lys, Cys, or Leu, Xaa²⁸ is Lys, Cys, or Pro, Xaa²⁹ is Lys, Cys, or Pro, Xaa³¹ is Lys, Cys, or Asn, Xaa³⁴ is Lys, Cys, or Ser, and Xaa³⁵ is Lys, Cys, or Asn, and X is substituted or unsubstituted amino, substituted or unsubstituted alkylamino, substituted or unsubstituted dialkylamino, substituted or unsubstituted cycloalkylamino, substituted or unsubstituted arylamino, substituted or unsubstituted aralkylamino, substituted or unsubstituted alkyloxy, substituted or unsubstituted aryloxy, substituted or unsubstituted aralkyloxy, hydroxyl, a bond to a duration enhancing moiety, or a linker to a duration enhancing moiety. The duration enhancing moiety can be covalently linked, optionally through a linker, to a side chain of a linking amino acid residue, X′ or X. The duration enhancing moiety can be covalently linked, optionally through a linker, to a backbone atom of the polypeptide component.

In another aspect, there is provided a pharmaceutical composition which includes a compound as described herein in combination with a pharmaceutically acceptable excipient.

In another aspect, there is provided a method for treating obesity, diabetes, or other metabolic disorder in a subject. The method includes administering a compound or pharmaceutical composition as described herein to a subject in need of treatment in an amount effective to treat the obesity, diabetes, or other metabolic disorder.

In yet another aspect, there is provided a method for the treatment in a subject in need of treatment for an eating disorder, insulin resistance, obesity, overweight, abnormal postprandial hyperglycemia, diabetes of any type including Type I, Type II and gestational diabetes, metabolic syndrome, dumping syndrome, hypertension, dyslipidemia, cardiovascular disease, hyperlipidemia, sleep apnea, cancer, pulmonary hypertension, cholescystitis, osteoarthritis, or short bowel syndrome. The method includes administering to a subject in need of treatment a compound or pharmaceutical composition described herein in an effective amount to treat the disease or disorder.

### BRIEF DESCRIPTION OF THE DRAWINGS

FIG. 1A depicts the daily cumulative body weight gain results as described herein for Cmpds 21, 25, 24, 22, 26 and vehicle. FIG. 1B depicts the daily food intake results for Cmpds 21, 25, 24, 22, 26 and vehicle. Legend: Cmpd 21 (box); Cmpd 25 (triangle tip up); Cmpd 24 (triangle tip down); Cmpd 22 (diamond); Cmpd 26 (open circle); vehicle (filled circle).

FIG. 2A depicts the results of comparison of twice weekly SC dosing of Cmpd 26 and continuous dosing of Cmpd 1 for two weeks in DIO rats. Legend: Vehicle (filled circle); Cmpd 26 (triangle); Cmpd 1 (box).

FIG. 2B depicts the results of comparison of once weekly SC dosing of Cmpd 23 and continuous infusion of Cmpd 1 for four weeks in DIO rats. Legend: Vehicle (filled circle); Cmpd 23 (triangle); Cmpd 1 (box).

FIG. 3A depicts the daily cumulative body weight gain results from a dose response study for Cmpd 23. FIG. 3B depicts the daily food intake results from the dose response study for Cmpd 23. Legend: vehicle (box); 12 nmol/kg (triangle tip up); 25 nmol/kg (triangle tip down); 50 nmol/kg (diamond); 125 nmol/kg (filled circle); 250 nmol/kg (open box).

FIG. 4A depicts the cumulative body weight gain results from a dose response study for Cmpd 19. Legend: vehicle (box); 50 nmol/kg (triangle tip up); 50 nmol/kg (triangle tip down); 50 nmol/kg (diamond).

FIG. 4B depicts the daily cumulative body weight gain results as described herein for Cmpds 23, 27 and vehicle. Legend: vehicle (dark filled box); Cmpd 23 (light filled box); Cmpd 27 (triangle).

FIG. 5A depicts the daily cumulative body weight gain results as described herein for Cmpds 26, 28, 29, 30 and vehicle. FIG. 5B depicts the daily food intake results for Cmpds 26, 28, 29, 30 and vehicle. Legend: Cmpd 26 (triangle tip down); Cmpd 28 (diamond); Cmpd 29 (large filled circle); Cmpd 30 (open box); vehicle (small filled circle).

FIG. 6A depicts the daily cumulative body weight gain results from a dose response study for Cmpd 29, and in comparison to Cmpd 31. FIG. 6B depicts the daily food intake results for Cmpd 29, and in comparison to Cmpd 31. Legend: Cmpd 31, 250 nmol/kg (box); Cmpd 29, 250 nmol/kg (triangle tip up); Cmpd 29, 125 nmol/kg (triangle tip down); Cmpd 29, 62.5 nmol/kg (diamond); Cmpd 29, 31.25 nmol/kg (large filled circle); vehicle (small filled circle).

FIG. 7A depicts the daily cumulative body weight gain results as described herein for Cmpds 29, 32, 33, 34, 35, 36 and vehicle. FIG. 7B depicts the daily food intake results for Cmpds 29, 32, 33, 34, 35, 36 and vehicle. Legend: Cmpd 29 (square); Cmpd 32 (diamond); Cmpd 33 (filled circle); Cmpd 34 (square); Cmpd 35 (triangle tip up); Cmpd 36 (triangle tip down); vehicle (small filled circle).

FIG. 8A depicts the daily cumulative body weight gain results as described herein for Cmpds 29, 41, 42 and vehicle. FIG. 8B depicts the daily food intake results for Cmpds 29, 41, 42 and vehicle. Legend: Cmpd 29 (square); Cmpd 41 (triangle tip up); Cmpd 42 (triangle tip down); vehicle (small filled circle).

### DETAILED DESCRIPTION OF THE INVENTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched chain, or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, consisting of at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two heteroatoms may be consecutive, such as, for example, —CH₂—NH—OCH₃.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

The term “oxo,” as used herein, means an oxygen that is double bonded to a carbon atom.

The term “alkylsulfonyl,” as used herein, means a moiety having the formula —S(O₂)—R′, where R′ is an alkyl group as defined above. R′ may have a specified number of carbons (e.g., “C₁-C₄ alkylsulfonyl”).

Each of the above terms (e.g., “alkyl,” “heteroalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN, and —NO₂ in a number ranging from zero to (2 m′+1), where m′ is the total number of carbon atoms in such radical. R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN, —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₙ—X′—(C″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A)-OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from:
   (i) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, and heteroaryl, substituted with at least one substituent selected from:
      (a) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, or heteroaryl, substituted with at least one substituent selected from: oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalkyl.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

Thus, the compounds of the present invention may exist as salts, such as with pharmaceutically acceptable acids. The present invention includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, the present invention provides compounds in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, tautomers, geometric isomers, and individual isomers are encompassed within the scope of the present invention. The compounds of the present invention do not include those that are known in the art to be too unstable to synthesize and/or isolate.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

The symbol “ [symbol] ” denotes the point of attachment of a chemical moiety to the remainder of a molecule or chemical formula.

“Ortholog” and like terms in the context of peptides refer to two or more peptide gene products wherein the genes coding the ortholog have evolved from a common ancestor, as known in the art.

“Analog” as used herein in the context of polypeptides refers to a compound that has insertions, deletions and/or substitutions of amino acids relative to a parent compound. An analog may have superior stability, solubility, efficacy, half-life, and the like. In some embodiments, an analog is a compound having at least 50%, for example 50%, 55%, 60%, 65%, 70%, 75%, 80%, 85%, 90%, 95%, 98%, or even higher, sequence identity to the parent compound.

The terms “identity,” “sequence identity” and the like in the context of comparing two or more nucleic acids or polypeptide sequences, refer to two or more sequences or subsequences that are the same or have a specified percentage of amino acid residues or nucleotides that are the same (i.e., about 50% identity, preferably 50%, 55%, 60%, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a sequence comparison algorithms as known in the art, for example BLAST or BLAST 2.0. This definition includes sequences that have deletions and/or additions, as well as those that have substitutions, as well as naturally occurring, e.g., polymorphic or allelic variants, and man-made variants. In preferred algorithms, account is made for gaps and the like, as known in the art. For sequence comparison, typically one sequence acts as a reference sequence, to which test sequences are compared. When using a sequence comparison algorithm, test and reference sequences are entered into a computer, subsequence coordinates are designated if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters. Optimal alignment of sequences for comparison can be conducted, e.g., by the local homology algorithm of Smith & Waterman, 1981, Adv. Appl. Math. 2:482, by the homology alignment algorithm of Needleman & Wunsch, 1970, J. Mol. Biol. 48:443, by the search for similarity method of Pearson & Lipman, 1988, Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection. See e.g., Current Protocols in Molecular Biology (Ausubel et al., eds. 1995 supplement)). Preferred examples of algorithms that are suitable for determining percent sequence identity and sequence similarity include the BLAST and BLAST 2.0 algorithms, which are described in Altschul et al., 1977, Nuci. Acids Res. 25:3389-3402 and Altschul et al., 1990, J. Mol. Biol. 215:403-410. BLAST and BLAST 2.0 are used, as known in the art, to determine percent sequence identity for the nucleic acids and proteins of the invention. Software for performing BLAST analyses is publicly available through the web site of the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., Id.). These initial neighborhood word hits act as seeds for initiating searches to find longer HSPs containing them. The word hits are extended in both directions along each sequence for as far as the cumulative alignment score can be increased. Cumulative scores are calculated using, e.g., for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, 1989, Proc. Natl. Acad. Sci. USA 89:10915) alignments (B) of 50, expectation (E) of 10, M=5, N=−4, and a comparison of both strands.

To determine the percent identity or similarity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same or similar amino acid residue or nucleotide as the corresponding position in the second sequence, then the molecules are identical or similar at that position. The percent identity or similarity between the two sequences is a function of the number of identical or similar positions shared by the sequences (i.e., % identity=# of identical positions/total # of positions (e.g., overlapping positions)×100). The similarity of two amino acids can be assessed by a variety of methods known in the art. For example, nonpolar neutral residues (e.g., Ala, Cys, Gly, Ile, Leu, Met, Phe, Pro, Trp, Val) can be considered similar, as can in turn acidic charged polar (e.g., Glu, Asp), basic charged polar (e.g., Arg, His, Lys) and neutral polar (e.g., Asn, Gln, Ser, Thr, Tyr) residues.

Both identity and similarity may be readily calculated. For example, in calculating percent identity, only exact matches may be counted, and global alignments may be performed as opposed to local alignments. Methods commonly employed to determine identity or similarity between sequences include, e.g., those disclosed in Carillo et al., 1988, SIAM J. Applied Math. 48:1073. Exemplary methods to determine identity are designed to give the largest match between the sequences tested. Exemplary methods to determine identity and similarity are also provided in commercial computer programs. A particular example of a mathematical algorithm utilized for the comparison of two sequences is the algorithm of Karlin et al., 1990, Proc. Natl. Acad. Sci. USA 87:2264-2268, and as modified e.g., as in Karlin et al., 1993, Proc. Natl. Acad. Sci. USA 90:5873-5877. Such an algorithm is incorporated into the NBLAST and XBLAST programs of Altschul et al., 1990, J. Mol. Biol. 215:403-410. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search, which detects distant relationships between molecules. When utilizing BLAST, Gapped BLAST, and PSI-Blast programs, the default parameters of the respective programs (e.g., XBLAST and NBLAST) can be used, as known in the art. Additionally, the FASTA method (Atschul et al., 1990, Id.) can be used. Another particular example of a mathematical algorithm useful for the comparison of sequences is the algorithm of Myers et al., 1988, CABIOS 4:11-17. Such an algorithm is incorporated into the ALIGN program (version 2.0), which is part of the GCG sequence alignment software package (Devereux et al., 1984, Nucleic Acids Res. 12(1):387). Percent identity can be determined by analysis with the AlignX® module in Vector NTI® (Invitrogen; Carlsbad Calif.).

“Obesity” and “overweight” refer to mammals having a weight greater than normally expected, and may be determined by, e.g., physical appearance, body mass index (BMI) as known in the art, waist-to-hip circumference ratios, skinfold thickness, waist circumference, and the like. The Centers for Disease Control and Prevention (CDC) define overweight as an adult human having a BMI of 25 to 29.9; and define obese as an adult human having a BMI of 30 or higher. Additional metrics for the determination of obesity exist. For example, the CDC states that a person with a waist-to-hip ratio greater than 1.0 is overweight.

“Lean body mass” refers to the fat-free mass of the body, i.e., total body weight minus body fat weight is lean body mass. Lean body mass can be measured by methods such as hydrostatic weighing, computerized chambers, dual-energy X-ray absorptiometry, skin calipers, magnetic resonance imaging (MRI) and bioelectric impedance analysis (BIA) as known in the art.

“Mammal” refers to warm-blooded animals that generally have fur or hair, that give live birth to their progeny, and that feed their progeny with milk. Mammals include humans; companion animals (e.g., dogs, cats); farm animals (e.g., cows, horses, sheep, pigs, goats); wild animals; and the like. In one embodiment, the mammal is a female. In one embodiment, the mammal is a female human. In one embodiment, the mammal is a cat or dog. In one embodiment, the mammal is a diabetic mammal, e.g., a human having type 2 diabetes. In one embodiment, the mammal is an obese diabetic mammal, e.g., an obese mammal having type 2 diabetes.

“Amylin agonist compounds” include native amylin peptides, amylin analog peptides, and other compounds (e.g., small molecules) that have amylin agonist activity. The “amylin agonist compounds” can be derived from natural sources, can be synthetic, or can be derived from recombinant DNA techniques. Amylin agonist compounds have amylin agonist receptor binding activity and may comprise amino acids (e.g., natural, unnatural, or a combination thereof), peptide mimetics, chemical moieties, and the like. The skilled artisan will recognize amylin agonist compounds using amylin receptor binding assays or by measuring amylin agonist activity in soleus muscle assays. Amylin agonist compounds can have an IC₅₀ of about 200 nM or less, about 100 nM or less, or about 50 nM or less, in an amylin receptor binding assay, such as that described herein, in U.S. Pat. No. 5,686,411, and US Publication No. 2008/0176804, the disclosures of which are incorporated by reference herein in their entireties and for all purposes. The term “IC₅₀” refers in the customary sense to the half maximal inhibitory concentration of a compound inhibiting a biological or biochemical function. Accordingly, in the context of receptor binding studies, IC₅₀ refers to the concentration of a test compound which competes half of a known ligand from a specified receptor. Amylin agonist compounds can have an EC₅₀ of about 20 nM or less, about nM 15 or less, about nM 10 or less, or about nM 5 or less in a soleus muscle assay, such as that described herein and in U.S. Pat. No. 5,686,411. The term “EC₅₀” refers in the customary sense to the effective concentration of a compound which induces a response halfway between a baseline response and maximum response, as known in the art. Amylin agonist compound can have at least 90% or 100% sequence identity to [^{25,28,29}Pro] human-amylin (pramlintide). The amylin agonist compound can be a pep tide chimera of amylin (e.g., human amylin, rat amylin, and the like) and calcitonin (e.g., human calcitonin, salmon calcitonin, and the like). Suitable and exemplary amylin agonist compounds are also described in US Publication No. 2008/0274952, the disclosure of which is incorporated by reference herein in its entirety and for all purposes. Unless indicated differently, the term “about” in the context of a numeric value refers to +/−10% of the numeric value.

“Fragment” in the context of polypeptides refers herein in the customary chemical sense to a portion of a polypeptide. For example, a fragment can result from N-terminal deletion or C-terminal deletion of one or more residues of a parent polypeptide, and/or a fragment can result from internal deletion of one or more residues of a parent polypeptide. The term “parent” in the context of polypeptides refers, in the customary sense, to a polypeptide which serves as a reference structure prior to modification, e.g., insertion, deletion and/or substitution. The terms “conjugate,” “peptide conjugate,” “polypeptide conjugate” and the like in the context of compounds useful in the methods described herein refer to component polypeptides which are bound to one or more duration enhancing moieties, optionally through a linker.

The terms “peptide” and “polypeptide” in the context of polypeptide components of the polypeptide conjugates described herein are synonymous. The term “peptide” refers in the customary sense to a polymer of amino acids connected by amide bonds. The terms “des-amino acid,” “des-AA,” “desLys” and the like refer to the absence of the indicated amino acid, as customary in the art. An amino acid (or functionality) being “absent” means that the residue (or functionality) formerly attached at the N-terminal and C-terminal side of the absent amino acid (or functionality) have become bonded together. The terms “peptide component” and “polypeptide component” refer to polypeptides included within a polypeptide conjugate described herein.

“Derivative” in the context of a polypeptide refers to a molecule having the amino acid sequence of a parent or analog thereof, but additionally having a chemical modification of one or more of its amino acid side groups, α-carbon atoms, backbone nitrogen atoms, terminal amino group, or terminal carboxylic acid group. A chemical modification includes, but is not limited to, adding chemical moieties, creating new bonds, and removing chemical moieties. Modifications at amino acid side groups include, but are not limited to, acylation of lysine ε-amino groups, N-alkylation of arginine, histidine, or lysine, alkylation of glutamic or aspartic carboxylic acid groups, and deamidation of glutamine or asparagine. Modifications of the terminal amino include, but are not limited to, the desamino, N-lower alkyl, N-di-lower alkyl, constrained alkyls (e.g. branched, cyclic, fused, adamantyl) and N-acyl modifications. Modifications of the terminal carboxy group include, but are not limited to, the amide, lower alkyl amide, constrained alkyls (e.g. branched, cyclic, fused, adamantyl)alkyl, dialkyl amide, and lower alkyl ester modifications. Furthermore, one or more side groups, or terminal groups, may be protected by protective groups known to the ordinarily-skilled synthetic chemist. The alpha-carbon of an amino acid may be mono- or dimethylated. Derivatives of the polypeptide components described herein are also contemplated wherein the stereochemistry of individual amino acids may be inverted from (L)/S to (D)/R at one or more specific sites. Also contemplated are polypeptide components modified by glycosylation, at e.g., Asn, Ser and/or Thr residues. Compounds useful in the methods provided may also be biologically active fragments of the peptides (native, agonist, analog, and derivative) herein described.

The terms “mimetic,” “peptidomimetic” and the like refer in the customary sense to a compound containing non-peptidic structural elements that is capable of agonizing or antagonizing the biological action(s) of a natural parent peptide.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the singular form “a”, “an”, and “the” includes plural references unless otherwise indicated or clear from context. For example, as will be apparent from context, “a” analog can include one or more analogs.

### II. Compounds

In a first aspect, there is provided a polypeptide conjugate which includes a polypeptide component to which one or more duration enhancing moieties are linked, optionally through a linker. Thus, the polypeptide component serves as a template (“polypeptide template”) to which is attached, preferably by covalent attachment, one or more duration enhancing moieties. Linkage of the duration enhancing moiety to the polypeptide component can be through a linker as described herein. Alternatively, linkage of the duration enhancing moiety to the polypeptide component can be via a direct covalent bond. The duration enhancing moiety can be a water soluble polymer as described herein. In some embodiments, a plurality of duration enhancing moieties are attached to the polypeptide component, in which case each linker to each duration enhancing moiety is independently selected from the linkers described herein.

In some embodiments, the polypeptide component includes an amino acid sequence of residues 1-37 of Formula (I) following, wherein up to 25% of the amino acids set forth in Formula (I) may be deleted or substituted with a different amino acid:

X′-Xaa¹-Cys²-Asn³-Thr⁴-Ala⁵-Thr⁶-Cys⁷-Ala⁸-Thr⁹-Gln¹⁰-Arg¹¹-Leu¹²-Ala¹³-Asn¹⁴-Phe¹⁵-Leu¹⁶-Val¹⁷-Xaa¹⁸-Ser¹⁹-Ser²⁰-Xaa²¹-Asn²²-Phe²³-Xaa²⁴-Xaa²⁵-Xaa²⁶-Xaa²⁷-Xaa²⁸-Xaa²⁹-Thr³⁰-Xaa³¹-Val³²-Gly³³-Xaa³⁴-Xaa³⁵-Thr³⁶-Tyr³⁷-X (SEQ ID NO: 4) (I).

In Formula (I), X′ is hydrogen, an N-terminal capping group, a bond to a duration enhancing moiety, or a linker to a duration enhancing moiety. Xaa¹ is Lys or a bond, Xaa¹⁸ is Lys, Cys, or His, Xaa²¹ is Lys, Cys, or Asn, Xaa²⁴ is Lys, Cys, or Gly, Xaa²⁵ is Lys, Cys, or Pro, Xaa²⁶ is Lys, Cys, or Ile, Xaa²⁷ is Lys, Cys, or Leu, Xaa²⁸ is Lys, Cys, or Pro, Xaa²⁹ is Lys, Cys, or Pro, Xaa³¹ is Lys, Cys, or Asn, Xaa³⁴ is Lys, Cys, or Ser, and Xaa³⁵ is Lys, Cys, or Asn. A person having ordinary skill in the art will immediately recognize that the polypeptide component of Formula (I), and other formulae disclosed herein, has an appropriate valency in order to attach to one or more duration enhancing moieties. For example, where a single duration enhancing moiety is present, the polypeptide component of Formula (I) is a monovalent peptide, which valency attaches to the duration enhancing moiety, optionally through a linker. Accordingly, where two duration enhancing moietites are present, the polypeptide component of Formula (I) is a divalent peptide, and so forth.

Further regarding Formula (I), the variable X represents a C-terminal functionality (e.g., a C-terminal cap). X is substituted or unsubstituted amino, substituted or unsubstituted alkylamino, substituted or unsubstituted dialkylamino, substituted or unsubstituted cycloalkylamino, substituted or unsubstituted arylamino, substituted or unsubstituted aralkylamino, substituted or unsubstituted alkyloxy, substituted or unsubstituted aryloxy, substituted or unsubstituted aralkyloxy, hydroxyl, a bond to a duration enhancing moiety, or a linker to a duration enhancing moiety. In some embodiments, the duration enhancing moiety is covalently linked, optionally through a linker, to a side chain of a linking amino acid residue, X′ or X. In some embodiments, the duration enhancing moiety is covalently linked, optionally through a linker, to a backbone atom of the polypeptide component. If the C-terminal of the polypeptide component with the sequence of residues 1-37 of Formula (I) is capped with a functionality X, then X is preferably amine thereby forming a C-terminal amide. The N-terminal of polypeptide components described herein, including the polypeptide component according to Formula (I), can be covalently linked to a variety of functionalities including, but not limited to, the acetyl group. The term “N-terminal capping group” refers in the customary sense to a moiety covalently bonded to the N-terminal nitrogen of a polypeptide, e.g., substituted or unsubstituted acyl, substituted or unsubstituted acyloxy, Schiff's bases, and the like, as known in the art. In some embodiments, the N-terminal functionality X′ is an amine-protecting group as known in the art, preferably Fmoc.

In some embodiments, up to 5%, 10%, 15%, 20%, 25%, 30%, 35%, 40%, 45% or even 50% of the amino acids of residues 1-37 of Formula (I) are deleted or substituted in a polypeptide component according to Formula (I). In some embodiments, the polypeptide component has 0, 1, 2, 3, 4, 5, 6, 7, 8, 9, 10, 11, 12, 13, 14, 15 or even 16 amino acid substitutions relative to the amino acid sequence set forth in Formula (I).

In some embodiments, the polypeptide component of the polypeptide conjugate has a sequence which has a defined sequence identity with respect to the residues 1-37 of the amino acid sequence according to Formula (I).

In some embodiments, the sequence identity between a polypeptide component described herein and residues 1-37 of Formula (I) is 50%, 55%, 60%, 65%, 70%, 75%, 80%, 85%, 90%, 95% or even higher. In some embodiments, up to 50%, 45%, 40%, 35%, 30%, 25%, 20%, 15%, 10%, 5% or even less of the amino acids set forth in residues 1-37 of Formula (I) may be deleted or substituted with a different amino acid. In some embodiments, the sequence identity is within the range 75%-100%. In some embodiments, the sequence identity is within the range 75%-90%. In some embodiments, the sequence identity is within the range 80%-90%. In some embodiments, the sequence identity is at least 75%. In some embodiments, the polypeptide component of the conjugate has the sequence of residues 1-37 of Formula (I).

In some embodiments, the polypeptide component has the sequence of Cmpd 12. In some embodiments, the polypeptide component has the sequence of Cmpd 6. In some embodiments, the polypeptide component has one or more conservative amino acid substitutions with respect to the sequence of Formula (I). “Conservative amino acid substitution” refers in the customary sense to substitution of amino acids having similar biochemical properties at the side chain (e.g., hydrophilicity, hydrophobocity, charge type, van der Waals radius, and the like). “Non-conservative amino acid substitution” refers in the customary sense to substitution of amino acids having dissimilar biochemical properties at the side chain.

It is understood that in the calculation of sequence identity with respect to any of the polypeptide components set forth herein (e.g., as found in residues 1-37 of Formula (I)), the sequence to be compared is taken over the amino acids disclosed therein, irrespective of any N-terminal (i.e., X′) or C-terminal (i.e., X) functionality present. It is further understood that the presence of a duration enhancing moiety covalently linked to the side chain of an amino acid is immaterial to the calculation of sequence identity. For example, a lysine substituted at any position of Formula (I) and additionally bonded, optionally through a linker, with a duration enhancing moiety is a lysine for purposes of sequence identity calculation.

Polypeptides including the sequence of residues 1-37 of Formula (I) can be considered to be chimeric combinations of amylin and calcitonin, or analogs thereof. Amylin is a peptide hormone synthesized by pancreatic β-cells that is co-secreted with insulin in response to nutrient intake. The sequence of amylin is highly preserved across mammalian species, with structural similarities to calcitonin gene-related peptide (CGRP), the calcitonins, the intermedins, and adrenomedullin. The glucoregulatory actions of amylin complement those of insulin by regulating the rate of glucose appearance in the circulation via suppression of nutrient-stimulated glucagon secretion and slowing gastric emptying. In insulin-treated patients with diabetes, pramlintide, a synthetic and equipotent analogue of human amylin, reduces postprandial glucose excursions by suppressing inappropriately elevated postprandial glucagon secretion and slowing gastric emptying. The sequences of rat amylin, human amylin and pramlintide follow, respectively:

(SEQ ID NO: 1)
KCNTATCATQRLANFLVRSSNNLGPVLPPTNVGSNTY;
(SEQ ID NO: 2)
KCNTATCATQRLANFLVHSSNNFGAILSSTNVGSNTY;
(SEQ ID NO: 3)
KCNTATCATQRLANFLVHSSNNFGPILPPTNVGSNTY.

In another aspect, there is provided a polypeptide conjugate, which is a derivative of pramlintide with SEQ ID NO:3 or an analog thereof, wherein the amino acid residue in position 1 is absent (i.e., des-Lys¹) and an amino acid residue in position 2 to 37 has been substituted with a lysine residue or cysteine residue and wherein said lysine residue or cysteine residue is linked to a polyethylene glycol polymer, optionally via a linker, wherein the amino acid numbering conforms with the amino acid number in SEQ ID NO:3.

In another aspect, the invention relates to a polypeptide conjugate, which is a derivative of pramlintide with SEQ ID NO:3 or an analog thereof, wherein the amino acid residue in position 1 is absent (i.e., des-Lys¹) and wherein an amino acid residue in any one of position 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 31, 32, 33, 34, 35, 36, or 37 is substituted with a lysine residue and wherein said lysine residue is linked to a polyethylene glycol polymer, optionally via a linker.

In another aspect, the invention relates to a polypeptide conjugate, which is a derivative of pramlintide with SEQ ID NO:3 or an analog thereof, wherein the amino acid residue in position 1 is absent (i.e., des-Lys¹) and wherein an amino acid residue in any one of position 18, 21, 24-29, 31, 34 or 35 is substituted with a lysine residue and wherein said lysine residue is linked to a polyethylene glycol polymer, optionally via a linker.

In another aspect, the invention relates to a polypeptide conjugate, which is a derivative of pramlintide with SEQ ID NO:3 or an analog thereof, wherein the amino acid residue in position 1 is absent (i.e., des-Lys¹) and wherein an amino acid residue in position 18 is substituted with a lysine residue and wherein said lysine residue is linked to a polyethylene glycol polymer, optionally via a linker.

In another aspect, the invention relates to a polypeptide conjugate, which is a derivative of pramlintide with SEQ ID NO:3 or an analog thereof, wherein the amino acid residue in position 1 is absent (i.e., des-Lys¹) and wherein an amino acid residue in position 21 is substituted with a lysine residue and wherein said lysine residue is linked to a polyethylene glycol polymer, optionally via a linker.

In another aspect, the invention relates to a polypeptide conjugate, which is a derivative of pramlintide with SEQ ID NO:3 or an analog thereof, wherein the amino acid residue in position 1 is absent (i.e., des-Lys¹) and wherein an amino acid residue in position 24 is substituted with a lysine residue and wherein said lysine residue is linked to a polyethylene glycol polymer, optionally via a linker.

In another aspect, the invention relates to a polypeptide conjugate, which is a derivative of pramlintide with SEQ ID NO:3 or an analog thereof, wherein the amino acid residue in position 1 is absent (i.e., des-Lys¹) and wherein an amino acid residue in position 25 is substituted with a lysine residue and wherein said lysine residue is linked to a polyethylene glycol polymer, optionally via a linker.

In another aspect, the invention relates to a polypeptide conjugate, which is a derivative of pramlintide with SEQ ID NO:3 or an analog thereof, wherein the amino acid residue in position 1 is absent (i.e., des-Lys¹) and wherein an amino acid residue in position 26 is substituted with a lysine residue and wherein said lysine residue is linked to a polyethylene glycol polymer, optionally via a linker.

In another aspect, the invention relates to a polypeptide conjugate, which is a derivative of pramlintide with SEQ ID NO:3 or an analog thereof, wherein the amino acid residue in position 1 is absent (i.e., des-Lys¹) and wherein an amino acid residue in position 27 is substituted with a lysine residue and wherein said lysine residue is linked to a polyethylene glycol polymer, optionally via a linker.

In another aspect, the invention relates to a polypeptide conjugate, which is a derivative of pramlintide with SEQ ID NO:3 or an analog thereof, wherein the amino acid residue in position 1 is absent (i.e., des-Lys¹) and wherein an amino acid residue in position 28 is substituted with a lysine residue and wherein said lysine residue is linked to a polyethylene glycol polymer, optionally via a linker.

In another aspect, the invention relates to a polypeptide conjugate, which is a derivative of pramlintide with SEQ ID NO:3 or an analog thereof, wherein the amino acid residue in position 1 is absent (i.e., des-Lys¹) and wherein an amino acid residue in position 29 is substituted with a lysine residue and wherein said lysine residue is linked to a polyethylene glycol polymer, optionally via a linker.

In another aspect, the invention relates to a polypeptide conjugate, which is a derivative of pramlintide with SEQ ID NO:3 or an analog thereof, wherein the amino acid residue in position 1 is absent (i.e., des-Lys¹) and wherein an amino acid residue in position 31 is substituted with a lysine residue and wherein said lysine residue is linked to a polyethylene glycol polymer, optionally via a linker.

In another aspect, the invention relates to a polypeptide conjugate, which is a derivative of pramlintide with SEQ ID NO:3 or an analog thereof, wherein the amino acid residue in position 1 is absent (i.e., des-Lys¹) and wherein an amino acid residue in position 34 is substituted with a lysine residue and wherein said lysine residue is linked to a polyethylene glycol polymer, optionally via a linker.

In another aspect, the invention relates to a polypeptide conjugate, which is a derivative of pramlintide with SEQ ID NO:3 or an analog thereof, wherein the amino acid residue in position 1 is absent (i.e., des-Lys¹) and wherein an amino acid residue in position 35 is substituted with a lysine residue and wherein said lysine residue is linked to a polyethylene glycol polymer, optionally via a linker.

Linkers.

The terms “linker” and the like, in the context of attachment of duration enhancing moieties to a polypeptide component in a polypeptide conjugate described herein, means a divalent species (-L-) covalently bonded in turn to a polypeptide component having a valency available for bonding and to a duration enhancing moiety having a valency available for bonding. The available bonding site on the polypeptide component is conveniently a side chain residue (e.g., lysine, cysteine, aspartic acid, and homologs thereof). In some embodiments, the available bonding site on the polypeptide component is the side chain of a lysine or a cysteine residue. In some embodiments, the available bonding site on the polypeptide component is the N-terminal amine. In some embodiments, the available bonding site on the polypeptide component is the C-terminal carboxyl. In some embodiments, the available bonding site on the polypeptide component is a backbone atom thereof. As used herein, the term “linking amino acid residue” means an amino acid within residues 1-37 of Formula (I) to which a duration enhancing moiety is attached, optionally through a linker.

In some embodiments, compounds are provided having a linker covalently linking a polypeptide component with a duration enhancing moiety. The linker is optional; i.e., any linker may simply be a bond. In some embodiments, the linker is attached at a side chain of the polypeptide component. In some embodiments, the linker is attached to a backbone atom of the polypeptide component.

In one embodiment, the linker is a polyfunctional amino acid, for example but not limited to, lysine and homologs thereof, aspartic acid and homologs thereof, and the like. The term “polyfunctional” in the context of an amino acid refers to a side chain functionality which can react to form a bond, in addition to the alpha amine and carboxyl functionalities of the amino acid. Exemplary functionalities of polyfunctional amino acids include, but are not limited to, amine, carboxyl and sulfhydryl functionalities.

In some embodiments, the linker comprises from 1 to 30 amino acids (“peptide linker”) linked by peptide bonds. The amino acids can be selected from the 20 naturally occurring amino acids. Alternatively, non-natural amino acids can be incorporated either by chemical synthesis, post-translational chemical modification or by in vivo incorporation by recombinant expression in a host cell. Some of these linker amino acids may be glycosylated. In another embodiment the 1 to 30 amino acids are selected from glycine, alanine, proline, asparagine, glutamine, and lysine. In some embodiments, the linker is made up of a majority of amino acids that are sterically unhindered, such as glycine, alanine and/or serine. Polyglycines are particularly useful, e.g. (Gly)₃, (Gly)₄ (SEQ ID NO: 5), (Gly)₅ (SEQ ID NO: 6), as are polyalanines, poly(Gly-Ala) and poly(Gly-Ser). Other specific examples of linkers are (Gly)₃Lys(Gly)₄ (SEQ ID NO: 7); (Gly)₃AsnGlySer(Gly)₂ (SEQ ID NO: 8); (Gly)₃Cys(Gly)₄ (SEQ ID NO: 9); and GlyProAsnGlyGly (SEQ ID NO: 10). Combinations of Gly and Ala are particularly useful as are combination of Gly and Ser. Thus in a further embodiment the peptide linker is selected from the group consisting of a glycine rich peptide, e.g. Gly-Gly-Gly; the sequences [Gly-Ser]ₙ (SEQ ID NO: 11), [Gly-Gly-Ser]ₙ (SEQ ID NO: 12), [Gly-Gly-Gly-Ser]ₙ (SEQ ID NO: 13) and [Gly-Gly-Gly-Gly-Ser]ₙ (SEQ ID NO: 14), where n is 1, 2, 3, 4, 5 or 6, for example [Gly-Gly-Gly-Gly Ser]₃ (SEQ ID NO: 15).

In some embodiments, the linker includes a divalent heteroatom. In some embodiments, the linker is, or includes, —O—, —S—, —S—S—, —OCO—, —OCONH—, and —NHCONH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. Representative linkers include —O—, —S—, —S—S—, —OCO—, —OCONH—, and —NHCONH—, amide and/or urethane attached to the duration enhancing moiety and the polypeptide component.

In some embodiments, the linker results from direct chemical conjugation between an amino acid side chain of a backbone functionality (moiety) of the polypeptide component and a functionality on the duration enhancing moiety. Exemplary of this type of bonding is the formation of an amide bond achieved by standard solid-phase synthetic methods, as well known in the art. The linkers described herein are exemplary, and linkers within the scope of this invention may be much longer and may include other residues.

In some embodiments, the linker includes two or more of substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In some embodiments, the linker has the structure -L¹-L²-, wherein L¹ and L² are each independently a divalent heteroatom, —O—, —S—, —S—S—, —OCO—, —OCONH—, and —NHCONH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In some embodiments, L¹ and L² are each independently —OCO—(CH₂)ₙ—CO—, —O—(CH₂)ₙ—NHCO—, —O—(CH₂)ₙ—, —O—(CH₂)ₙ—CONH—(CH₂)ₙ—, —O—(CH₂)ₙ—, —SO₂—(CH₂)ₙ—, —SO₂—(CH₂)ₙ—,S—, wherein “n” is independently 1-5 at each occurrence.

In some embodiments, the linker has the structure —OCO—(CH₂)ₙ—CO—, —O—(CH₂)ₙ—NHCO—, —O—(CH₂)ₙ—, —O—(CH₂)ₙ—CONH—(CH₂)ₙ—, —O—(CH₂)ₙ—, —SO₂—(CH₂)ₙ—, —SO₂—(CH₂)ₙ—,S—, wherein “n” is independently 1-5 at each occurrence.

In some embodiments, a substituted group within a linker or a substituted linker group described herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted or unsubstituted cycloalkylene, substituted or unsubstituted heterocycloalkylene, substituted or unsubstituted arylene, or substituted or unsubstituted heteroarylene within a linker described herein is substituted with at least one substituent group. In other embodiments, at least one or all of these groups are substituted with at least one size-limited substituent group. Alternatively, at least one or all of these groups are substituted with at least one lower substituent group.

In other embodiments of the linkers described herein, each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, each substituted or unsubstituted heterocycloalkyl is a substituted or unsubstituted 4 to 8 membered heterocycloalkyl, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene substituted or unsubstituted C₄-C₈ cycloalkylene, and each substituted or unsubstituted heterocycloalkylene is a substituted or unsubstituted 4 to 8 membered heterocycloalkylene.

Alternatively, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, each substituted or unsubstituted heterocycloalkyl is a substituted or unsubstituted 5 to 7 membered heterocycloalkyl, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene substituted or unsubstituted C₅-C₆ cycloalkylene, and each substituted or unsubstituted heterocycloalkylene is a substituted or unsubstituted 5 to 7 membered heterocycloalkylene.

Polypeptide Component.

Polypeptide components useful in the compounds and methods described herein include, but are not limited to, the polypeptide components set forth in residues 1-37 of Formula (I) provided in Table 1 below. Unless indicated to the contrary, all peptides described herein, including peptides having an expressly provided sequence, are contemplated in both free carboxylate and amidated forms.

TABLE 1
Component polypeptides useful in the
compounds described herein.

| | | SEQ |
|---|---|---|
| | | ID |
| Cmpd | Description (sequence) | NO: |
| 1 | KCNTATCATQRLANFLVRSSNNLGPVLPPTNVGSNTY- | 16 |
| | NH₂ | |
| 2 | CNTATCATQRLANFLVRSSNNLGPVLPPTNVGSNTY-NH₂ | 17 |
| | ([desLys¹]-Cmpd 1) | |
| 3 | KCNTATCATQRLANFLVRSSKNLGPVLPPTNVGSNTY- | 18 |
| | NH₂ | |
| 4 | CNTATCATQRLANFLVRSSKNLGPVLPPTNVGSNTY-NH₂ | 19 |
| | ([desLys¹]-Cmpd 3) | |
| 5 | KCNTATCATQRLANFLVRSSNNLGPKLPPTNVGSNTY- | 20 |
| | NH₂ | |
| 6 | CNTATCATQRLANFLVRSSNNLGPKLPPTNVGSNTY-NH₂ | 21 |
| | ([desLys¹]-Cmpd 5) | |
| 7 | KCNTATCATQRLANFLVRSSNNLGPVLPPTKVGSNTY- | 22 |
| | NH₂ | |
| 8 | CNTATCATQRLANFLVRSSNNLGPVLPPTKVGSNTY-NH₂ | 23 |
| | ([desLys¹]-Cmpd 7) | |
| 9 | KCNTATCATQRLANFLVHSSNNFGPILPPTNVGSNTY- | 24 |
| | NH₂ | |
| 10 | CNTATCATQRLANFLVHSSNNFGPILPPTNVGSNTY-NH₂ | 25 |
| | ([desLys¹]-Cmpd 9) | |
| 11 | CNTATCATQRLANFLVHSSKNFGPILPPTNVGSNTY-NH₂ | 26 |
| 12 | CNTATCATQRLANFLVHSSNNFGPKLPPTNVGSNTY-NH₂ | 27 |
| 13 | CNTATCATQRLANFLVHSSNNFGPILPPTKVGSNTY-NH₂ | 28 |
| 14 | CNTATCATQRLANFLVHSSNNFKPILPPTNVGSNTY-NH₂ | 29 |
| 15 | CNTATCATQRLANFLVHSSNNFGKILPPTNVGSNTY-NH₂ | 30 |
| 16 | CNTATCATQRLANFLVHSSNNFGPIKPPTNVGSNTY-NH₂ | 31 |
| 17 | CNTATCATQRLANFLVHSSNNFGPILKPTNVGSNTY-NH₂ | 32 |
| 18 | CNTATCATQRLANFLVHSSNNFGPILPKTNVGSNTY-NH₂ | 33 |
| 37 | CNTATCATQRLANFLVKSSNNFGPILPPTNVGSNTY-NH₂ | 34 |
| 38 | CNTATCATQRLANFLVHSSNNFGPILPPTNVGKNTY-NH₂ | 35 |
| 39 | CNTATCATQRLANFLVHSSNNFGPILPPTNVGSKTY-NH₂ | 36 |

Duration Enhancing Moieties.

In some embodiments, the duration enhancing moiety is included within a “linked duration enhancing moiety” with formula -L-R, wherein R is a duration enhancing moiety as described herein, and L is a linker or a bond. Where L is a linker, L can be —C(O)—, —NH—, —O—, —S—, —S—S—, —OCO—, —OCONH—, —NHCONH—, substituted or unsubstituted alkylene, substituted or unsubstituted alkenylene, substituted or unsubstituted urethane, substituted or unsubstituted alkylamide, substituted or unsubstituted alkylsulfone, substituted or unsubstituted heteroalkylene, substituted or unsubstituted cycloalkylene, substituted or unsubstituted heterocycloalkylene, substituted or unsubstituted arylene, or substituted or unsubstituted heteroarylene, and the like, as known in the art.

In some embodiments, L is R¹-substituted or unsubstituted alkylene, R¹-substituted or unsubstituted alkenylene, R¹-substituted or unsubstituted urethane, R¹-substituted or unsubstituted alkylamide, R¹-substituted or unsubstituted alkylsulfone, R¹-substituted or unsubstituted heteroalkylene, R¹-substituted or unsubstituted cycloalkylene, R¹-substituted or unsubstituted heterocycloalkylene, substituted or unsubstituted arylene, or substituted or unsubstituted heteroarylene. R¹ is R²-substituted or unsubstituted alkyl, R²-substituted or unsubstituted heteroalkyl, R²-substituted or unsubstituted cycloalkyl, R²-substituted or unsubstituted heterocycloalkyl, R²-substituted or unsubstituted aryl, or R²-substituted or unsubstituted heteroaryl. R² is R³-substituted or unsubstituted alkyl, R³-substituted or unsubstituted heteroalkyl, R³-substituted or unsubstituted cycloalkyl, R³-substituted or unsubstituted heterocycloalkyl, R³-substituted or unsubstituted aryl, or R³-substituted or unsubstituted heteroaryl. R³ is unsubstituted alkyl, unsubstituted heteroalkyl, unsubstituted cycloalkyl, unsubstituted heterocycloalkyl, unsubstituted aryl or unsubstituted heteroaryl.

In some embodiments, the linked duration enhancing moiety -L-R is covalently bonded to an amino acid side chain of the polypeptide component, or to a backbone atom or moiety thereof. Exemplary backbone moieties include a free amine at the N-terminal, and a free carboxyl or carboxylate at the C-terminal. In some embodiments, an amino acid side chain or a backbone atom or moiety is covalently bonded to a polyethylene glycol or a derivative thereof.

Water-Soluble Polymers.

In some embodiments, the duration enhancing moiety R is a water-soluble polymer. A “water soluble polymer” means a polymer which is sufficiently soluble in water under physiologic conditions of e.g., temperature, ionic concentration and the like, as known in the art, to be useful for the methods described herein. A water soluble polymer can increase the solubility of a peptide or other biomolecule to which such water soluble polymer is attached. Indeed, such attachment has been proposed as a means for improving the circulating life, water solubility and/or antigenicity of administered proteins, in vivo. See e.g., U.S. Pat. No. 4,179,337; U.S. Published Appl. No. 2008/0032408. Many different water-soluble polymers and attachment chemistries have been used towards this goal, such as polyethylene glycol, copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and the like.

Polyethylene Glycols.

In some embodiments, the linked duration enhancing moiety -L-R includes a polyethylene glycol. Polyethylene glycol (“PEG”) has been used in efforts to obtain therapeutically usable polypeptides. See e.g., Zalipsky, S., 1995, Bioconjugate Chemistry, 6:150-165; Mehvar, R., 2000, J. Pharm. Pharmaceut. Sci., 3:125-136. As appreciated by one of skill in the art, the PEG backbone [(CH₂CH₂—O—)ₙ, n: number of repeating monomers] is flexible and amphiphilic. Without wishing to be bound by any theory or mechanism of action, the long, chain-like PEG molecule or moiety is believed to be heavily hydrated and in rapid motion when in an aqueous medium. This rapid motion is believed to cause the PEG to sweep out a large volume and prevents the approach and interference of other molecules. As a result, when attached to another chemical entity (such as a peptide), PEG polymer chains can protect such chemical entity from immune response and other clearance mechanisms. As a result, pegylation can lead to improved drug efficacy and safety by optimizing pharmacokinetics, increasing bioavailability, and decreasing immunogenicity and dosing frequency. “Pegylation” refers in the customary sense to conjugation of a PEG moiety with another compound. For example, attachment of PEG has been shown to protect proteins against proteolysis. See e.g., Blomhoff, H. K. et al., 1983, Biochim Biophys Acta, 757:202-208. Unless expressly indicated to the contrary, the terms “PEG,” “polyethylene glycol polymer” and the like refer to polyethylene glycol polymer and derivatives thereof, including methoxy-PEG (mPEG).

A variety of means have been used to attach polymer moieties such as PEG and related polymers to reactive groups found on the protein. See e.g., U.S. Pat. No. 4,179,337; U.S. Pat. No. 4,002,531; Abuchowski et al., 1981, in “Enzymes as Drugs,” J. S. Holcerberg and J. Roberts, (Eds.), pp. 367-383; Zalipsky, S., 1995, Bioconjugate Chemistry, 6:150-165. The use of PEG and other polymers to modify proteins has been discussed. See e.g., Cheng, T.-L. et al., 1999m, Bioconjugate Chem., 10:520-528; Belcheva, N. et al., 1999, Bioconjugate Chem., 10:932-937; Bettinger, T. et al., 1998, Bioconjugate Chem., 9:842-846; Huang, S.-Y. et al., 1998, Bioconjugate Chem., 9:612-617; Xu, B. et al. 1998, Langmuir, 13:2447-2456; Schwarz, J. B. et al., 1999, J. Amer. Chem. Soc., 121:2662-2673; Reuter, J. D. et al., 1999, Bioconjugate Chem., 10:271-278; Chan, T.-H. et al., 1997, J. Org. Chem., 62:3500-3504. Typical attachment sites in proteins include primary amino groups, such as those on lysine residues or at the N-terminus, thiol groups, such as those on cysteine side-chains, and carboxyl groups, such as those on glutamate or aspartate residues or at the C-terminus. Common sites of attachment are to the sugar residues of glycoproteins, cysteines or to the N-terminus and lysines of the target polypeptide. The terms “pegylated” and the like refer to covalent attachment of polyethylene glycol to a polypeptide or other biomolecule, optionally through a linker as described herein and/or as known in the art.

In some embodiments, a PEG moiety in a polypeptide conjugate described herein has a nominal molecular weight within a specified range. As customary in the art, the size of a PEG moiety is indicated by reference to the nominal molecular weight, typically provided in kilodaltons (kD). The molecular weight is calculated in a variety of ways known in the art, including number, weight, viscosity and “Z” average molecular weight. It is understood that polymers, such as PEG and the like, exist as a distribution of molecule weights about a nominal average value.

Exemplary of the terminology for molecular weight for PEGs, the term “mPEG40 KD” refers to a methoxy polyethylene glycol polymer having a nominal molecular weight of 40 kilodaltons. Reference to PEGs of other molecular weights follows this convention. In some embodiments, the PEG moiety has a nominal molecular weight in the range 10-100 KD, 20-80 KD, 20-60 KD, or 20-40 KD. In some embodiments, the PEG moiety has a nominal molecular weight of 10, 15, 20, 25, 30, 35, 40, 45, 50, 55, 60, 65, 70, 75, 80, 85, 90, 95 or even 100 KD. Preferably, the PEG moiety has a molecular weight of 20, 25, 30, 40, 60 or 80 KD.

PEG molecules useful for derivatization of polypeptides are typically classified into linear, branched and Warwick (i.e., PolyPEG®) classes of PEGs, as known in the art. Unless expressly indicated to the contrary, the PEG moieties described herein are linear PEGs. Furthermore, the terms “two arm branched,” “Y-shaped” and the like refer to branched PEG moieties, as known in the art. The term “Warwick” in the context of PEGs, also known as “comb” or “comb-type” PEGs, refers to a variety of multi-arm PEGs attached to a backbone, typically poly(methacrylate), as known in the art. Regarding nomenclature including conventions employed in the table provided herein, absent indication to the contrary a PEG moiety is attached to the backbone of the peptide. For example, Cmpd 19 is the result of the conjugation of mPEG40 KD to the N-terminal nitrogen of Cmpd 1. Similarly, Cmpd 20 is the result of conjugation of mPEG40 KD to the N-terminal nitrogen of Cmpd 2. Standard single letter abbreviations for amino acids can be used, as can standard three-letter abbreviations. For example, Cmpd 24 is an analog of Cmpd 10 wherein the residue at position 26 of Cmpd 9 is substituted for lysine, and the pendant amine functionality of lysine 26 (i.e., K²⁶) is conjugated with a PEG40 KD moiety. Exemplary compounds are provided in Table 2 below.

TABLE 2
Pegylated compounds

| | | SEQ ID |
|---|---|---|
| Cmpd | Description | NO: |
| 19 | mPEG40KD-Cmpd 1 | 37 |
| 20 | mPEG40KD-Cmpd 2 | 38 |
| 21 | [K²¹(mPEG40KD)]-Cmpd 3 | 39 |
| 22 | [K²¹(mPEG40KD)]-Cmpd 4 | 40 |
| 23 | [K²⁶(mPEG40KD)]-Cmpd 5 | 41 |
| 24 | [K²⁶(mPEG40KD)]-Cmpd 6 | 42 |
| 25 | [K³¹(mPEG40KD)]-Cmpd 7 | 43 |
| 26 | [K³¹(mPEG40KD)]-Cmpd 8 | 44 |
| 27 | [K²⁶(Y-shaped-mPEG4OKD)]-Cmpd 5 | 45 |
| 28 | [K²¹(mPEG40KD)]-Cmpd 11 | 46 |
| 29 | [K²⁶(mPEG40KD)]-Cmpd 12 | 47 |
| 30 | [K³¹(mPEG40KD)]-Cmpd 13 | 48 |
| 31 | [K²⁶(Y-shaped-mPEG40KD)]-Cmpd 12 | 49 |
| 32 | [K²⁴(mPEG40KD)]-Cmpd 14 | 50 |
| 33 | [K²⁵(mPEG40KD)]-Cmpd 15 | 51 |
| 34 | [K²⁷(mPEG40KD)]-Cmpd 16 | 52 |
| 35 | [K²⁸(mPEG40KD)]-Cmpd 17 | 53 |
| 36 | [K²⁹(mPEG40KD)]-Cmpd 18 | 54 |
| 40 | [K¹⁸(mPEG40KD)]-Cmpd 37 | 55 |
| 41 | [K³⁴(mPEG40KD)]-Cmpd 38 | 56 |
| 42 | [K³⁵(mPEG40KD)]-Cmpd 39 | 57 |

Recombinant PEG.

In some embodiments, the duration enhancing moiety -L-R conjugated with a polypeptide described herein includes an unstructured recombinant polypeptide. See e.g., Schellenberger et al., 2009, Nature Biotechnology, 27:1186-1192, incorporated herein by reference and for all purposes. The terms “recombinant PEG,” “rPEG,” “rPEG duration enhancing moiety” and the like refer to substantially unstructured recombinant polypeptide sequences which act as surrogates for PEG as duration enhancing moieties in conjugation with polypeptide components having a defined sequence identity relative to the amino acid sequence of Formula (I). rPEGs and polypeptide conjugates thereof have the potentially significant advantage that synthesis can be achieved by recombinant methods, not requiring the solid-phase or solution-phase chemical synthetic steps of, for example but not limited to, conjugation of PEG with the polypeptide.

It has been found that stable, highly expressed, unstructured polypeptides can be conjugated with biologically active molecules, which results in modulation of a variety of biological parameters, including but not limited to, serum half-life. For example, by exclusively incorporating A, E, G, P, S and T, Schellenberger et al (Id.) disclose that the apparent half-lives of conjugates with exenatide, green fluorescent protein (GFP) and human growth hormone (hGH) are significantly increased relative to the unconjugated polypeptides.

In some embodiments, the rPEG duration enhancing moiety does not include a hydrophobic residue (e.g., F, I, L, M, V, W or Y), a side chain amide-containing residue (e.g., N or Q) or a positively charged side chain residue (e.g., H, K or R). In some embodiments, the rPEG duration enhancing moiety includes A, E, G, P, S or T. In some embodiments, the rPEG includes glycine at 10-20%, 20-30%, 30-40%, 40-50%, 50-60%, 60-70%, 70-80%, 80-90%, 90-99%, or even glycine at 100%.

In embodiments where the rPEG duration enhancing moiety is conjugated at the N-terminal or C-terminal of the polypeptide which is at least 75% identical to the structure of Formula (I), the conjugated polypeptide and rPEG are synthesized by recombinant methods known in the art. In embodiments where the rPEG duration enhancing moiety is conjugated at a side chain of the polypeptide which is at least 75% identical to the structure of Formula (I), the rPEG moiety is synthesized by recombinant methods and subsequently conjugated to the polypeptide by methods known in the art and disclosed herein.

Chemical Substitution.

In some embodiments, each substituted group in a polypeptide conjugate described herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted or unsubstituted cycloalkylene, substituted or unsubstituted heterocycloalkylene, substituted or unsubstituted arylene, or substituted or unsubstituted heteroarylene described herein is substituted with at least one substituent group. In some embodiments, at least one or all of these groups are substituted with at least one size-limited substituent group. In some embodiments, at least one or all of these groups are substituted with at least one lower substituent group.

In some embodiments, each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, each substituted or unsubstituted heterocycloalkyl is a substituted or unsubstituted 4 to 8 membered heterocycloalkyl, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene substituted or unsubstituted C₄-C₈ cycloalkylene, and each substituted or unsubstituted heterocycloalkylene is a substituted or unsubstituted 4 to 8 membered heterocycloalkylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, each substituted or unsubstituted heterocycloalkyl is a substituted or unsubstituted 5 to 7 membered heterocycloalkyl, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene substituted or unsubstituted C₅-C₆ cycloalkylene, and each substituted or unsubstituted heterocycloalkylene is a substituted or unsubstituted 5 to 7 membered heterocycloalkylene.

### III. Exemplary Syntheses

General Methods of Polypeptide Synthesis.

The polypeptide components of the polypeptide conjugates described herein may be prepared using biological, chemical, and/or recombinant DNA techniques that are known in the art. Exemplary methods are described herein and in U.S. Pat. No. 6,872,700; WO 2007/139941; WO 2007/140284; WO 2008/082274; WO 2009/011544; and US Publication No. 2007/0238669, the disclosures of which are incorporated herein by reference in their entireties and for all purposes. Other methods for preparing the compounds are set forth herein and/or known in the art.

For example, the polypeptide components of the compounds described herein may be prepared using standard solid-phase peptide synthesis techniques, such as an automated or semiautomated peptide synthesizer. Typically, using such techniques, an alpha-N-carbamoyl protected amino acid and an amino acid attached to the growing peptide chain on a resin are coupled at room temperature in an inert solvent (e.g., dimethylformamide, N-methylpyrrolidinone, methylene chloride, and the like) in the presence of coupling agents (e.g., dicyclohexylcarbodiimide, 1-hydroxybenzo-triazole, and the like) in the presence of a base (e.g., diisopropylethylamine, and the like). The alpha-N-carbamoyl protecting group is removed from the resulting peptide-resin using a reagent (e.g., trifluoroacetic acid, piperidine, and the like) and the coupling reaction repeated with the next desired N-protected amino acid to be added to the peptide chain. Suitable N-protecting groups are well known in the art, such as t-butyloxycarbonyl (tBoc) fluorenylmethoxycarbonyl (Fmoc), and the like. The solvents, amino acid derivatives and 4-methylbenzhydryl-amine resin used in the peptide synthesizer may be purchased from a variety of commercial sources, including for example Applied Biosystems Inc. (Foster City, Calif.).

For chemical synthesis, solid phase peptide synthesis can be used for the polypeptide conjugates, since in general solid phase synthesis is a straightforward approach with excellent scalability to commercial scale, and is generally compatible with relatively long polypeptide conjugates. Solid phase peptide synthesis may be carried out with an automatic peptide synthesizer (Model 430A, Applied Biosystems Inc., Foster City, Calif.) using the NMP/HOBt (Option 1) system and tBoc or Fmoc chemistry (See Applied Biosystems User's Manual for the ABI 430A Peptide Synthesizer, Version 1.3B Jul. 1, 1988, section 6, pp. 49-70, Applied Biosystems, Inc., Foster City, Calif.) with capping. Boc-peptide-resins may be cleaved with HF (−5° C. to 0° C., 1 hour). The peptide may be extracted from the resin with alternating water and acetic acid, and the filtrates lyophilized. The Fmoc-peptide resins may be cleaved according to standard methods (e.g., Introduction to Cleavage Techniques, Applied Biosystems, Inc., 1990, pp. 6-12). Peptides may be also assembled using an Advanced Chem Tech Synthesizer (Model MPS 350, Louisville, Ky.).

Amine Pegylation.

Covalent attachment of PEG can be conveniently achieved by a variety of methods available to one skilled in the synthetic chemical arts. For pegylation at backbone or side chain amine, PEG reagents are typically reacted under mild conditions to afford the pegylated compound. Optionally, additional steps including but not limited to reduction are employed. In a typical peptide-mPEG conjugation scheme, N-hydroxylsuccinimide (NHS) functionalized mPEG can be mixed with peptide having a free amine in a suitable solvent (e.g., dry DMF) under nitrogen in the presence of DIPEA (e.g., 3 equivalents per TFA counterion) for a suitable time (e.g., 24 hrs). The conjugate can be precipitated by the addition of a precipitation reagent (e.g., cold diethyl ether). The precipitate can be isolated by centrifugation and dissolved in water followed by lyophilization. Purification can be afforded by a variety of chromatographic procedures (e.g., MacroCap SP cation exchange column using gradient 0.5 M NaCl). Purity can be checked by SDS-PAGE. Mass spectrometry (e.g., MALDI) can be used to characterize the conjugate after dialysis against water.

PEG-SS (Succinimidyl Succinate).

PEG-SS reacts with amine groups under mild conditions to form the amide, as shown in Scheme 1. NHS functionalization provides amino reactive PEG derivatives that can react with primary amine groups at pH 7˜9 to form stable amide bonds. Reaction can be finished in 1 hour or even less time. Exemplary reactions follow in Schemes 1 and 2.

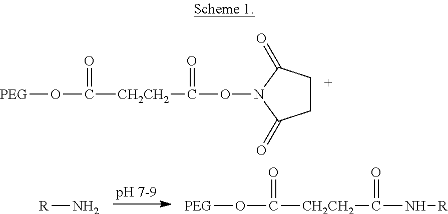

PEG-SG (Succinimidyl Glutarate).

Similarly, PEG-SG reacts with amine groups to form the corresponding amide, as shown in Scheme 2.

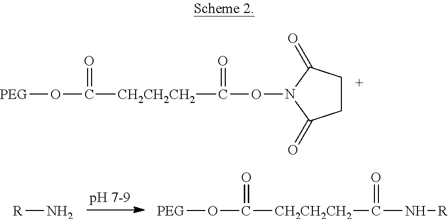

PEG-NPC (p-Nitrophenyl Carbonate).

PEG-NPC reacts with amine functionalities to form the relatively stable urethane functionality, as shown in Scheme 3.

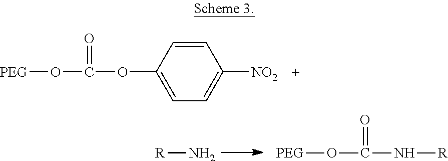

PEG-Isocyanate.

As shown in Scheme 4, PEG-isocyanate can react with amine to form the resultant relatively stable urethane linkage.

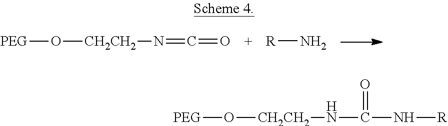

PEG-Aldehyde.

A variety of PEG-aldehyde reactions with amine can afford the imine, which can be further reduced to afford the pegylated amine. The reaction pH may be important for target selectivity. N-terminal amine pegylation may be at around pH 5. For example, reaction of mPEG-propionaldehyde with peptide amine, followed by reduction affords the compound depicted in Scheme 5 following.

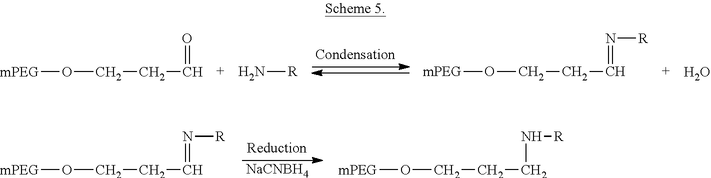

Similarly, condensation of mPEG-amide-propionaldehyde with amine and subsequent reduction can afford the compounds depicted in Scheme 6 following.

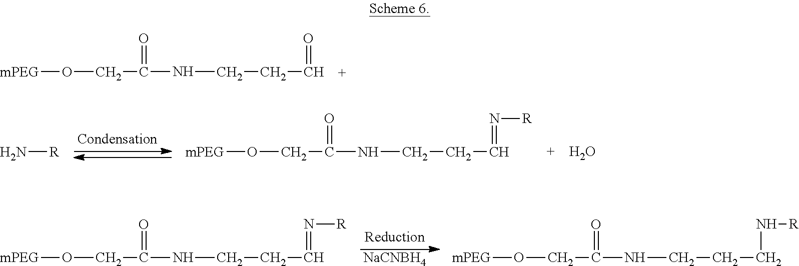

Reaction of mPEG-urethane-propionaldehyde with amine and subsequent reduction can afford the compounds depicted in Scheme 7 following.

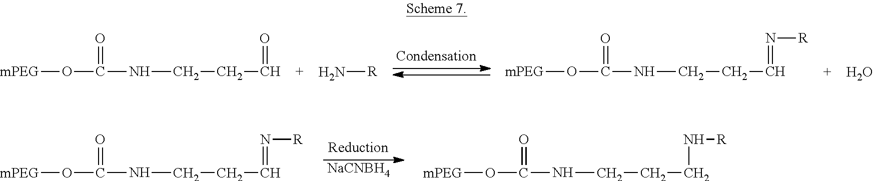

Furthermore, reaction of mPEG-butylaldehyde with amine and subsequent reduction can afford the compounds depicted in Scheme 8 following.

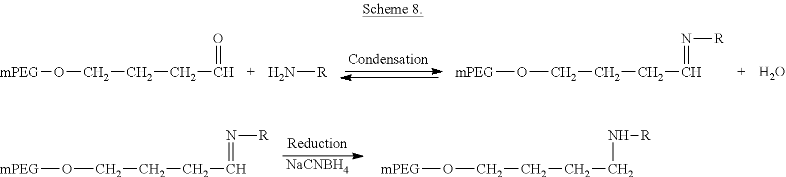

Thiol Pegylation: PEG-Maleimide.

Pegylation is conveniently achieved at free thiol groups by a variety of methods known in the art. For example, as shown in Scheme 9 following, PEG-maleimide pegylates thiols of the target compound in which the double bond of the maleimic ring breaks to connect with the thiol. The rate of reaction is pH dependent and best conditions are found around pH 8.

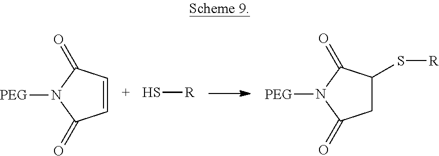

PEG-Vinylsulfone.

Additionally, as depicted in Scheme 10 following, PEG-vinylsulfone is useful for the pegylation of free thiol.

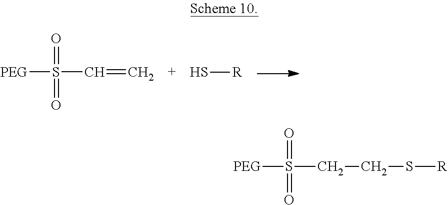

PEG-Orthopyridyl-Disulfide (OPSS).

Formation of disulfide linked PEG to a polypeptide is achieved by a variety of methods known in the art, including the reaction depicted in Scheme 11 following. In this type of linkage, the resulting PEG conjugate can be decoupled from the polypeptide by reduction with, for example but not limited to, borohydride, small molecule dithiol (e.g., dithioerythritol) and the like.

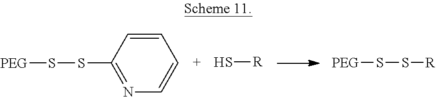

PEG-Iodoacetamide.

PEG-iodoacetamide pegylates thiols to form stable thioether bonds in mild basic media. This type of conjugation presents an interesting aspect in that by strong acid analysis the pegylated cysteine residue of the protein can give rise to carboxymethylcysteine which can be evaluated by a standard amino acid analysis (for example, amino acid sequencing), thus offering a method to verify the occurrence of the reaction. A typical reaction scheme is depicted in Scheme 12 following.

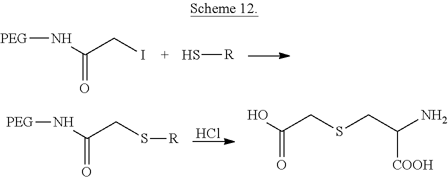

Purification of Compounds.

Purification of compounds described herein generally follows methods available to the skilled artisan. In a typical purification procedure, a crude peptide-PEG conjugate is initially purified via ion exchange chromatography, e.g., Macro Cap SP cation exchanger column. A typical purification procedure employs Buffer A (20 mM sodium acetate buffer, pH 5.0) and Buffer B (20 mM sodium acetate buffer, pH 5.0, 0.5 M sodium chloride) in a gradient elution program, e.g., 0-0% Buffer B (20 min), followed by 0-50% Buffer B (50 min), then 100% Buffer B (20 min). The flow rate is typically 3 mL/min. SDS polyacrylamide gel visualization of the collected fractions is conducted, followed by dialysis against water of the suitable fraction pool and lyophilization of the resultant. Analytical characterization typically employs MALDI mass spectroscopy.

### IV. Methods of Use

In one aspect, there is provided a method for the treatment in a subject in need of treatment for metabolic disorders such as, but not limited to obesity, diabetes (e.g., type 2 or non-insulin dependent diabetes, type 1 diabetes, and gestational diabetes), dyslipidemia, eating disorders, insulin-resistance syndrome, and/or cardiovascular disease. In another aspect, there is provided a method for the treatment in a subject in need of treatment for abnormal postprandial hyperglycemia, dumping syndrome, hypertension, hyperlipidemia, sleep apnea, cancer, pulmonary hypertension, cholescystitis, osteoarthritis, and short bowel syndrome. The method includes administering to a subject in need of treatment an effective amount of a compound or pharmaceutical composition described herein.

As used herein, a “subject” may include any mammal, including but not limited to rats, mice and humans. A “subject” also includes domestic animals (e.g., dogs, cats, horses), as well as other animals. Subjects may have at least one of the metabolic disorders described herein. Subjects can be of any age. Accordingly, these disorders can be found in young adults and adults (defined herein as those aged 65 or under) as well as infants, children, adolescents, and the elderly (defined herein as over the age of 65). In fact, certain segments of the population may be particularly prone to having a particular condition, such as eating disorders in adolescents and young adults. The elderly may be particularly susceptible to conditions such as depression.

As used herein, and as well-understood in the art, “treatment” is an approach for obtaining beneficial or desired results, including clinical results. “Treating,” “palliating,” or “ameliorating” a disease, disorder, or condition means that the extent, undesirable clinical manifestations of a condition, or both, of a disorder or a disease state are lessened and/or the time course of the progression is slowed (i.e., lengthened in time), as compared to not treating the disorder. For purposes of the methods disclosed herein, beneficial or desired clinical results include, but are not limited to, alleviation or amelioration of one or more symptoms, diminishment of extent of disorder, stabilized (i.e., not worsening) state of disorder, delay or slowing of disorder progression, amelioration or palliation of the disorder, and remission (whether partial or total), whether detectable or undetectable. “Treatment” can also mean prolonging survival as compared to expected survival if not receiving treatment. Further, treating does not necessarily occur by administration of one dose, but often occurs upon administration of a series of doses. Thus, a “therapeutically effective amount,” an amount sufficient to palliate, or an amount sufficient to treat a disease, disorder, or condition may be administered in one or more administrations.

Obesity and its associated disorders including overweight are common and serious public health problems in the United States and throughout the world. Upper body obesity is the strongest risk factor known for type 2 diabetes mellitus and is a strong risk factor for cardiovascular disease. Obesity is a recognized risk factor for hypertension, atherosclerosis, congestive heart failure, stroke, gallbladder disease, osteoarthritis, sleep apnea, reproductive disorders such as polycystic ovarian syndrome, cancers of the breast, prostate, and colon, and increased incidence of complications of general anesthesia. See, e.g., Kopelman, 2000, Nature 404:635-43.

Obesity reduces life-span and carries a serious risk of the co-morbidities listed above, as well disorders such as infections, varicose veins, acanthosis nigricans, eczema, exercise intolerance, insulin resistance, hypertension hypercholesterolemia, cholelithiasis, orthopedic injury, and thromboembolic disease. See e.g., Rissanen et al, 1990, Br. Med. J., 301:835-7. Obesity is also a risk factor for the group of conditions called insulin resistance syndrome, or “Syndrome X” and metabolic syndrome. The worldwide medical cost of obesity and associated disorders is enormous.

The pathogenesis of obesity is believed to be multi-factoral. A problem is that, in obese subjects, nutrient availability and energy expenditure do not come into balance until there is excess adipose tissue. The central nervous system (CNS) controls energy balance and coordinates a variety of behavioral, autonomic and endocrine activities appropriate to the metabolic status of the animal. The mechanisms or systems that control these activities are broadly distributed across the forebrain (e.g., hypothalamus), hindbrain (e.g., brainstem), and spinal cord. Ultimately, metabolic (i.e., fuel availability) and cognitive (i.e., learned preferences) information from these systems is integrated and the decision to engage in appetitive (food seeking) and consummatory (ingestion) behaviors is either turned on (meal procurement and initiation) or turned off (meal termination). The hypothalamus is thought to be principally responsible for integrating these signals and then issuing commands to the brainstem. Brainstem nuclei that control the elements of the consummatory motor control system (e.g., muscles responsible for chewing and swallowing). As such, these CNS nuclei have literally been referred to as constituting the “final common pathway” for ingestive behavior.

Neuroanatomical and pharmacological evidence support that signals of energy and nutritional homeostasis integrate in forebrain nuclei and that the consummatory motor control system resides in brainstem nuclei, probably in regions surrounding the trigeminal motor nucleus. There are extensive reciprocal connection between the hypothalamus and brainstem. A variety of CNS-directed anti-obesity therapeutics (e.g., small molecules and peptides) focus predominantly upon forebrain substrates residing in the hypothalamus and/or upon hindbrain substrates residing in the brainstem.

Insulin resistance is a major pathophysiological feature in both obese and non-obese type 2 diabetics, and was previously believed to be due mainly to a post-binding defect in insulin action. See e.g., Berhanu et al., 1982, J. Clin. Endoc. Metab. 55:1226-1230. Such a defect could be due to an intrinsic property of peripheral cells, or caused by a change in concentration of a humoral factor in plasma, or both. Previous attempts at demonstrating a humoral factor responsible for insulin resistance have yielded conflicting results. Nor has it been possible to demonstrate an intrinsic post-binding defect in insulin resistance in type 2 diabetes mellitus (see: Howard, B. V. Diabetes 30: 562-567 (1981); Kolterman, O. G. et al., J. Clin. Invest.: 68: 957-969 (1981)).

The mechanisms of insulin resistance in type 2 diabetes are complex. Evidence, gleaned mainly from studies on adipose tissue, was said to suggest that in the mildest cases, insulin resistance could be accounted for largely by a deficiency in numbers of insulin receptors on peripheral target cells, but that as the degree of fasting hyperglycaemia increases, a postreceptor defect of insulin action emerges and progressively increases in significance (see: Kolterman et al., supra). The impaired glucose tolerance accompanying insulin resistance in type 2 diabetes is believed to be caused largely by decreased glucose uptake in perpheral tissues, but incomplete glucose-induced suppression of hepatic glucose production has also been said to be implicated. See e.g., Wajngot et al., 1982, Proc. Natl. Acad. Sci. USA 70:4432-4436. In both obese and non-obese type 2 diabetics, the insulin dose-response curve is shifted to the right and there is a marked decrease in the maximal rate of glucose disposal and of total-body glucose metabolism in type 2 diabetics compared with non-diabetic subjects (Kolterman et al., Id.; De Fronzo, R. A. et al., 1985, J. Clin. Invest. 76:149-155).

In another general aspect, the compounds of the invention may be useful for reducing food intake, reducing appetite, inducing satiety, reducing the nutrients available to the body to store as fat, causing weight loss, affecting body composition, altering body energy content or energy expenditure, improving lipid profile (including reducing LDL cholesterol and triglyceride levels and/or changing HDL cholesterol levels), slowing gastrointestinal motility, delay gastric emptying, moderating the postprandial blood glucose excursions, preventing or inhibiting glucagon secretion, and decreasing blood pressure.

Thus, in certain embodiments, the compounds of the invention are useful for treating or preventing conditions or disorders which can be alleviated by reducing the nutrients available to the body to store as fat. Such conditions and disorders include, but are not limited to, eating disorders, insulin-resistance, obesity, abnormal postprandial hyperglycemia, diabetes of any kind, including Type I, Type II, and gestational diabetes, Metabolic Syndrome, Dumping Syndrome, hypertension, dyslipidemia, cardiovascular disease, hyperlipidemia, sleep apnea, cancer, pulmonary hypertension, cholecystitis, and osteoarthritis.

Non-limiting examples of a cardiovascular condition or disease are hypertension, myocardial ischemia, and myocardial reperfusion. Compounds of the invention may also be useful in treating or preventing other conditions associated with obesity including stroke, cancer (e.g., endometrial, breast, prostate, and colon cancer), gallbladder disease, sleep apnea, reduced fertility, and osteoarthritis. In other embodiments, compounds of the invention may be used to alter body composition for aesthetic reasons, to enhance one's physical capabilities, or to produce a leaner meat source.

In another general aspect, compounds of the invention may be used to inhibit the secretion of ghrelin. Accordingly, compounds of the invention may be utilized to treat or prevent ghrelin related disorders such as Prader-Willi syndrome, diabetes of all types and its complications, obesity, hyperphagia, hyperlipidemia, or other disorders associated with hypernutrition.

In another general aspect, compounds of the invention may be useful for treating or preventing Barrett's esophagus, Gastroesophageal Reflux Disease (GERD) and conditions associated therewith. Such conditions can include, but are not limited to, heartburn, heartburn accompanied by regurgitation of gastric/intestinal contents into the mouth or the lungs, difficulty in swallowing, coughing, intermittent wheezing and vocal cord inflammation (conditions associated with GERD), esophageal erosion, esophageal ulcer, esophageal stricture, Barrett's metaplasia (replacement of normal esophageal epithelium with abnormal epithelium), Barrett's adenocarcinoma, and pulmonary aspiration. Amylin and amylin agonists have anti-secretory properties, such as inhibition of gastric acids, inhibition of bile acids, and inhibition of pancreatic enzymes. Moreover, amylin has been found to have a gastroprotective effect. Accordingly, these properties of amylin and amylin agonists may render them particularly useful in the treatment or prevention of Barrett's esophagus, and/or GERD and related or associated conditions as described herein.

In another general aspect, compounds of the invention may further be useful for treating or preventing pancreatitis, pancreatic carcinoma, and gastritis. Moreover, compounds of the invention may be useful in the treatment and prevention of pancreatitis in patients who have undergone endoscopic retrograde cholangiopancreatography (ERCP). It has further been discovered that amylin and amylin agonists may have a surprisingly superior therapeutic effect when combined with somatostatin. Accordingly, in certain embodiments, methods for treating or preventing pancreatitis comprise administering compounds of the invention and administering somatostatin and somatostatin agonists.

In another general aspect, compounds of the invention may also be useful for decreasing bone resorption, decreasing plasma calcium, and inducing an analgesic effect. Accordingly, compounds of the invention may be useful to treat bone disorder such as osteopenia and osteoporosis. In yet other embodiments, compounds of the invention may be useful to treat pain and painful neuropathy.

In another general aspect, compounds of the invention may also be useful for treating short bowel syndrome. Short bowel syndrome, or short gut syndrome, means a gastrointestinal syndrome characterized by symptoms resulting from the malabsorption of nutrients such as abdominal pain, diarrhea, fluid retention, unintended weight loss, and extreme fatigue due to an undeveloped bowel during gestation or following the surgical resection of a significant length of small bowel. Accordingly, as used herein, the term “short bowel syndrome” also includes short gut syndrome and massive small bowel resection. Intestinal hormonal reflexes and feedback loops can be disrupted leading to an increase in the volume of proximal gastric and small bowel sections and altered motility patterns. Water, sodium and magnesium losses can lead to electrolyte disturbances. Certain specific absorptive functions may also be impaired which are unique to certain parts of the intestine, such as the absorption of vitamin B12, bile salts and other fat soluble vitamins by the ileum. The compounds of the invention may provide substantial improvement in bowel habits, nutritional status and quality of life of short bowel syndrome patients, and further may reduce the need for parenteral nutrition and small bowel transplant.

### V. Assays

Methods for production and assay of compounds described herein are generally available to the skilled artisan. Representative assays for the compounds and methods described herein follow.

Food Intake.

Without wishing to be bound by any theory, it is believed that food intake is useful in the assessment of the utility of a compound as described herein. For example, it is known that a number of metabolic pathologies are related to food intake (e.g., diabetes, obesity). Accordingly, an initial screening can be conducted to determine the extent to which food intake is modulated by administration of compounds described herein, and a positive initial screening can be useful in subsequent development of a compound.

A variety of food intake assays are available to one of skill in the art. For example, in the so-called “home cage model” of food intake, subjects (e.g., rats) are maintained in their home cage, and food intake along with total weight of the subject is measured following injection of test compound. In the so-called “feeding patterns model” of food intake assay, subjects (e.g., rats) are habituated to a feeding chamber and to injections prior to testing. After test compound administration, the subjects are immediately placed into the feeding chamber, and food intake is automatically determined as a function of time (e.g., 1-min intervals). For both tests, the food is standard chow or any of a variety of chows (e.g., high fat) known in the art. In the so-called “mouse food intake” assay, a test compound may be tested for appetite suppression, or for an effect on body weight gain in diet-induced obesity (DIO) mice. In a typical mouse food intake assay, female NIH/Swiss mice (8-24 weeks old) are group housed with a 12:12 hour light:dark cycle with lights on at 0600. Water and a standard pelleted mouse chow diet are available ad libitum, except as noted. Animals are fasted starting at approximately 1500 hrs, 1 day prior to experiment. The morning of the experiment, animals are divided into experimental groups. In a typical study, n=4 cages with 3 mice/cage. At time=0 min, all animals are given an intraperitoneal injection of vehicle or compound, typically in an amount ranging from about 10 nmol/kg to 75 nmol/kg, and immediately given a pre-weighed amount (10-15 g) of the standard chow. Food is removed and weighed at various times, typically 30, 60, and 120 minutes, to determine the amount of food consumed. See e.g., Morley et al., 1994, Am. J. Physiol. 267:R178-R184). Food intake is calculated by subtracting the weight of the food remaining at the e.g. 30, 60, 120, 180 and/or 240 minute time point, from the weight of the food provided initially at time=0. Significant treatment effects are identified by ANOVA (p<0.05). Where a significant difference exists, test means are compared to the control mean using Dunnett's test (Prism v. 2.01, GraphPad Software Inc., San Diego, Calif.). For any test described herein, administration of test compound can be by any means, including injection (e.g., subcutaneous, intraperitoneal, and the like), oral, or other methods of administration known in the art.

In Vitro Assays.

Without wishing to be bound by any theory or mechanism of action, it is believed that correlations exist between the results of in vitro (e.g., receptor) assays, and the utility of agents for the treatment of metabolic diseases and disorders. Accordingly, in vitro assays (e.g., cell based assays) are useful as a screening strategy for potential metabolic agents, such as described herein. A variety of in vitro assays are known in the art, including those described as follows.

Calcitonin Adenylate Cyclase Assay (Functional Assay).

The calcitonin receptor mediated adenylate cyclase activation can be measured using an HTRF (Homogeneous Time-Resolved Fluorescence) cell-based cAMP assay kit from CisBio. This kit is a competitive immunoassay that uses cAMP labeled with the d2 acceptor fluorophore and an anti-cAMP monoclonal antibody labeled with donor Europium Cryptate. Increase in cAMP levels is registered as decrease in time-resolved fluorescence energy transfer between the donor and acceptor. Peptides can be serially diluted with buffer and transferred to, for example, a 384-well compound plate. C1a-HEK cells stably expressing the rat C1a calcitonin receptor can be detached from cell culture flasks and resuspended at 2×10⁶ cell/ml in stimulation buffer containing 500 μM IBMX, and d2 fluorophore at 1:40. Cells can be added to the compound plate at a density of 12,500 per well and incubated in the dark for 30 minutes at room temperature for receptor activation. Cells can be subsequently lysed by the addition of anti-cAMP Cryptate solution diluted with the kit conjugate/lysis buffer (1:40). After 1 to 24 hours incubation in the dark, the plate can be counted on a Tecan Ultra capable of measuring time-resolved fluorescence energy transfer.

Amylin Receptor Binding Assay.

RNA membranes can be incubated with approximately 20 pM (final concentration) of ¹²⁵I-rat amylin (Bolton-Hunter labeled, PerkinElmer, Waltham, Mass.) and increasing concentrations of test compound for 1 hour at ambient temperature in, for example, 96-well polystyrene plates. Bound fractions of well contents can be collected onto a 96 well glass fiber plate (pre-blocked for at least 30 minutes in 0.5% PEI (polyethyleneimine)) and washed with 1×PBS using a Perkin Elmer plate harvester. Dried glass fiber plates can be combined with scintillant and counted on a multi-well Perkin Elmer scintillation counter.

CGRP Receptor Binding Assay.

SK—N-MC cell membranes can be incubated with approximately 50 pM (final concentration) of ¹²⁵I-human CGRP (PerkinElmer, Waltham, Mass.) and increasing concentrations of test compound for 1 hour at ambient temperature in 96-well polystyrene plates. Bound fractions of well contents can be collected onto a 96 well glass fiber plate (pre-blocked for at least 30 minutes in 0.5% PEI) and washed with 1×PBS using a Perkin Elmer plate harvester. Dried glass fiber plates can be combined with scintillant and counted on a multiwell Perkin Elmer scintillation counter.

Calcitonin Receptor Binding Assay.

C1a-HEK cell membranes can be incubated with approximately 50 pM (final concentration) of ¹²⁵I-human calcitonin (PerkinElmer, Waltham, Mass.) and increasing concentrations of test compound for 1 hour at ambient temperature in, for example, 96-well polystyrene plates. Bound fractions of well contents can be collected onto a 96 well glass fiber plate (pre-blocked for at least 30 minutes in 0.5% PEI) and washed with 1×PBS using a Perkin Elmer plate harvester. Dried glass fiber plates can be combined with scintillant and counted on a multiwell Perkin Elmer scintillation counter.

### VI. Pharmaceutical Compositions

In one aspect, there is provided a pharmaceutical composition which includes a compound of the invention as described herein in combination with a pharmaceutically acceptable excipient.

A. Formulations

The compounds described herein can be prepared and administered in a wide variety of oral, parenteral, and topical dosage forms. Thus, the compounds of the present invention can be administered by injection (e.g. intravenously, intramuscularly, intracutaneously, subcutaneously, intraduodenally, or intraperitoneally). Also, the compounds described herein can be administered by inhalation, for example, intranasally. Additionally, the compounds of the present invention can be administered transdermally. It is also envisioned that multiple routes of administration (e.g., intramuscular, oral, transdermal) can be used to administer the compounds of the invention. Accordingly, the present invention also provides pharmaceutical compositions comprising a pharmaceutically acceptable carrier or excipient and one or more compounds of the invention.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substance that may also act as diluents, flavoring agents, binders, preservatives, tablet disintegrating agents, or an encapsulating material.

In powders, the carrier is a finely divided solid in a mixture with the finely divided active component. In tablets, the active component is mixed with the carrier having the necessary binding properties in suitable proportions and compacted in the shape and size desired.

The powders and tablets preferably contain from 5% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for the compounds of the invention are injectible, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampoules are convenient unit dosages. The compounds of the invention can also be incorporated into liposomes or administered via transdermal pumps or patches. Pharmaceutical admixtures suitable for use in the present invention include those described, for example, in PHARMACEUTICAL SCIENCES (17th Ed., Mack Pub. Co., Easton, Pa.) and WO 96/05309, the teachings of both of which are hereby incorporated by reference.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg, more typically 1.0 mg to 1000 mg, most typically 10 mg to 500 mg, according to the particular application and the potency of the active component. The composition can, if desired, also contain other compatible therapeutic agents.

Some compounds may have limited solubility in water and therefore may require a surfactant or other appropriate co-solvent in the composition. Such co-solvents include: Polysorbate 20, 60, and 80; Pluronic F-68, F-84, and P-103; cyclodextrin; and polyoxyl 35 castor oil. Such co-solvents are typically employed at a level between about 0.01% and about 2% by weight.

Viscosity greater than that of simple aqueous solutions may be desirable to decrease variability in dispensing the formulations, to decrease physical separation of components of a suspension or emulsion of formulation, and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents are typically employed at a level between about 0.01% and about 2% by weight.

The compositions of the present invention may additionally include components to provide sustained release and/or comfort. Such components include high molecular weight, anionic mucomimetic polymers, gelling polysaccharides, and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

B. Effective Dosages

Pharmaceutical compositions provided by the present invention include compositions wherein the active ingredient is contained in a therapeutically effective amount, i.e., in an amount effective to achieve its intended purpose. The actual amount effective for a particular application will depend, inter alia, on the condition being treated. For example, when administered in methods to treat a metabolic disease or disorder, such compositions will contain an amount of active ingredient effective to achieve the desired result (e.g. relieving the symptoms of the metabolic disease or disorder).

The dosage and frequency (single or multiple doses) of compound administered can vary depending upon a variety of factors, including route of administration; size, age, sex, health, body weight, body mass index, and diet of the recipient; nature and extent of symptoms of the disease being treated; presence of other diseases or other health-related problems; kind of concurrent treatment; and complications from any disease or treatment regimen. Other therapeutic regimens or agents can be used in conjunction with the methods and compounds of the invention.

For any compound described herein, the therapeutically effective amount can be initially determined from a variety of assays, including but not limited to cell culture assays and food intake assays. Target concentrations will be those concentrations of active compound(s) that are capable of eliciting a biological response in cell culture assay, or eliciting a food intake response.

Therapeutically effective amounts for use in humans may be determined from animal models. For example, a dose for humans can be formulated to achieve a concentration that has been found to be effective in animals. The dosage in humans can be adjusted by monitoring the underlying metabolic disease or disorder and adjusting the dosage upwards or downwards, as known in the art and/or as described herein.

Dosages may be varied depending upon the requirements of the patient and the compound being employed. The dose administered to a patient, in the context of the present invention, should be sufficient to effect a beneficial therapeutic response in the patient over time. The size of the dose also will be determined by the existence, nature, and extent of any adverse side effects. Generally, treatment is initiated with smaller dosages, which are less than the optimum dose of the compound. Thereafter, the dosage is increased by small increments until the optimum effect under circumstances is reached. In one embodiment of the invention, the dosage range is 0.001% to 10% w/v. In another embodiment, the dosage range is 0.1% to 5% w/v.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is entirely effective to treat the clinical symptoms demonstrated by the particular patient. This planning should involve the careful choice of active compound by considering factors such as compound potency, relative bioavailability, patient body weight, presence and severity of adverse side effects, preferred mode of administration, and the toxicity profile of the selected agent.

C. Toxicity

The ratio between toxicity and therapeutic effect for a particular compound is its therapeutic index and can be expressed as the ratio between LD₅₀ (the amount of compound lethal in 50% of the population) and ED₅₀ (the amount of compound effective in 50% of the population). Compounds that exhibit high therapeutic indices are preferred. Therapeutic index data obtained from cell culture assays and/or animal studies can be used in formulating a range of dosages for use in humans. The dosage of such compounds preferably lies within a range of plasma concentrations that include the ED₅₀ with little or no toxicity. The dosage may vary within this range depending upon the dosage form employed and the route of administration utilized. See, e.g. Fingl et al., In: THE PHARMACOLOGICAL BASIS OF THERAPEUTICS, Ch.1, p. 1, 1975. The exact formulation, route of administration, and dosage can be chosen by the individual physician in view of the patient's condition and the particular method in which the compound is used.

### VII. Examples

### Example 1

### Preparation of Compounds

Compounds of the invention were synthesized by several methods.
a) For example, Compound 19 was prepared by treating mPEG40K-aldehyde with the N-terminal of Compound 1 in a reductive alkylation reaction to generate specifically N-terminal pegylated Compound 1.
b) In another example, Compound 20 was prepared by reacting the N-terminal amino group of Compound 2 with an mPEG40K—NHS (n-hydroxysuccinimide ester).
c) In another example, Compounds 21, 23, 25, and 27 were prepared as follows:

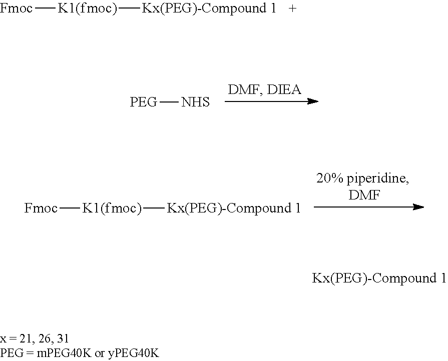

Analogs of Compound 1 with Fmoc protected lysine1 and a mutated lysine at 21, 26 and 31 positions were treated with mPEG40K—NHS in DMF with DIEA. The resulting pegylated peptide was deprotected by piperidine to give the pegylated free peptide.
d) In another example, Compounds 26, 22, and 24 were prepared by selective pegylation on a lysine side-chain. Analogs of Compound 2 with a mutated lysine at positions 21, 24-29 and 31 were treated with mPEG40K—NHS in DMF with DIEA. The crude product was purified and analyzed for regio-specificity.

### Example 2

### Receptor Binding Activity

Methods.

An amylin binding assay was performed in membranes prepared from the nucleus accumbens portion of the brain (rat), testing serial diluted peptide compounds described herein.

First, peptides were solvated in sterile distilled water at 200 μM concentration (where peptide weight is approx. 80%). Then peptides were diluted to a 2× starting concentration at 10⁻⁶ M with 1× buffer (20 mM HEPES, 5 mM MgCl₂, 1 mM CaCl₂, 0.5% BSA) and serially diluted with buffer using Perkin Elmer Multi-Probe II robot. Prepared membranes were diluted at (16-Fold) or at 32.5 μg/well and combined with 1× buffer or serial diluted with controls or peptide compounds and ¹²⁵I-rAmylin, respectively (Perkin Elmer Life Science, ID #I-3248). Plate was incubated for 1 hour at room temperature on a shaker. Plate was dried for 1.5 hours at 50° C., then overnight at room temperature. Scintillant was added (Microscint 20, Perkin Elmer Cat#6013621) and CPM determined by reading on a Perkin Elmer/Wallac TriLux multiwell scintillation counter capable of reading radiolabeled iodine.

Receptor binding activity can be expressed, for example in Table 3, as an IC₅₀ value, calculated from the raw data using an iterative curve-fitting program using a 4-parameter logistic equation (PRISM®, GraphPAD Software, La Jolla, Calif.), as known in the art.

Results.

As shown in Table 3 below, pegylated compounds of the invention demonstrate nanomolar binding activity at the amylin receptor.

TABLE 3
Receptor Binding Assay

| | Amylin binding |
|---|---|
| Compound | IC50 (nM) |
| 1 | 0.10 |
| 2 | 0.42 |
| 19 | 40 |
| 20 | 78 |
| 21 | 104 |
| 23 | 66 |
| 25 | 86 |
| 27 | 470 |
| 26 | 75 |
| 22 | 112 |
| 24 | 54 |
| 28 | 131 |
| 29 | 39 |
| 30 | 61 |
| 31 | 132 |
| 32 | 79 |
| 33 | 31 |
| 34 | 51 |
| 35 | 53 |
| 36 | 27 |

A second amylin receptor binding assay was performed to measure the potency of test compounds, e.g., polypeptides disclosed herein, in displacing ¹²⁵I-amylin (rat) from human amylin receptor 3 (AMY3) ectopically expressed in a cell line, e.g., a Codex ACTOne™ cell line. This cell line was generated using ACTOne™ HEK293-CNG-hCalcR cell line (CB-80200-258) stably expressing human RAMP3 (NCBI protein database CAA04474) to produce the human AMY3 receptor.

Crude membranes from AMY3 cell cultures were prepared by homogenization in ice cold 20 mM HEPES containing protease inhibitors (Roche Cat#11873580001). The crude membranes were incubated with 20 pM ¹²⁵I. (Perkin Elmer Cat#NEX4480) (2000 Ci/mmol) and increasing concentration of test peptide. Incubation was carried out in 20 mM HEPES with 5 mM MgCl₂ and 1 mM CaCl₂ for 60 minutes at ambient temperature in 96-well polystyrene plates (Costar Cat#3797). Incubations were terminated by rapid filtration through UniFilter® 96 plates GF/B (Perkin Elmer, Cat#6005199), pre-soaked for at least 30 minutes in 0.5% polyethylenimine. The Unifilter® plates were washed several times using ice cold PBS using a MicroMate 96 Cell Harvester (Perkin Elmer). Unifilter plates were dried, scintillant added (Microscint 20, Perkin Elmer Cat#6013621) and CPM determined by reading on a Perkin Elmer/Wallac TriLux multiwell scintillation counter capable of reading radiolabeled iodine.

The potency (IC₅₀) of test peptide was determined by the analysis of a concentration-response curve using non-linear regression analysis fitted to a 4-parameter curve. Binding affinities were calculated using GraphPad Prism® software (GraphPad Software, Inc., San Diego, Calif.). The results are shown in Table 4 below.

Results.

As shown in Table 4 below, pegylated compounds of the invention demonstrate nanomolar binding activity at the AMY3 receptor.

TABLE 4
Receptor Binding Assay

| | Amylin binding |
|---|---|
| Compound | IC50 (nM) |
| 1 | 0.247 |
| 28 | 388 |
| 29 | 60 |
| 30 | 80 |
| 31 | 1132 |
| 32 | 111 |
| 33 | 48 |
| 34 | 87 |
| 35 | 46 |
| 36 | 47 |
| 40 | 238 |
| 41 | 77 |
| 42 | 143 |

### Example 3

### Amylin Functional Assay

Method.

This assay is used to measure increases in cyclic-AMP (cAMP) in the Codex ACTOne™ cell line via the peptide-induced activation of over expressed human Amylin 3 receptor (hAMY3, Gs coupled). Accumulation of cAMP was measured following 30′ peptide treatment using the HTRF (CisBio) cell-based cAMP assay kit in 384-well format. Efficacy of peptides was determined relative to cell treatment with 10 uM forskolin (a constitutive activator of adenylate cyclase), and potency (EC₅₀) of peptides was determined by the analysis of a concentration-response curve using non-linear regression analysis fitted to a 4-parameter model.

Results.

As shown in Table 5 below, pegylated compounds of the invention demonstrate subnanomolar to nanomolar functional activity at the AMY3 receptor.

TABLE 5
Amylin FunctionalAssay

| Compound | EC50 (nM) |
|---|---|
| 1 | 0.005 |
| 28 | 2.577 |
| 29 | 0.114 |
| 30 | 0.145 |
| 31 | 2.560 |
| 32 | 0.598 |
| 33 | 0.126 |
| 34 | 0.296 |
| 35 | 0.295 |
| 36 | 0.159 |
| 40 | 0.85 |
| 41 | 0.034 |
| 42 | 0.122 |

### Example 4

### Effect of Pegylation on Food Intake: Cmpds 21, 25, 24, 22, 26

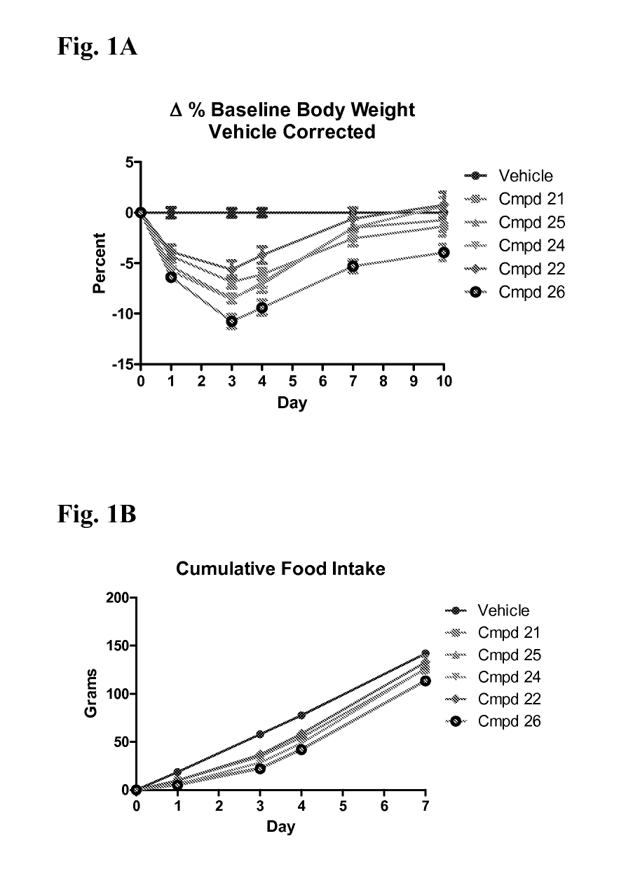

Lean rats were administered a subcutaneous (SC) once weekly injection of test compound or vehicle. FIGS. 1A-1B provides the result of a multi-day food intake assay. The effect on 24-hour food intake was investigated for Cmpds 21, 25, 24, 22, and 26, using vehicle as control. The results of FIGS. 1A-B demonstrate that each of the tested compounds was efficacious in reducing body weight and food intake for three days. In the case of some of the compounds, weight loss was still evident even after one week.

### Example 5

### Effect of Pegylation on Food Intake: Cmpds 26, 23

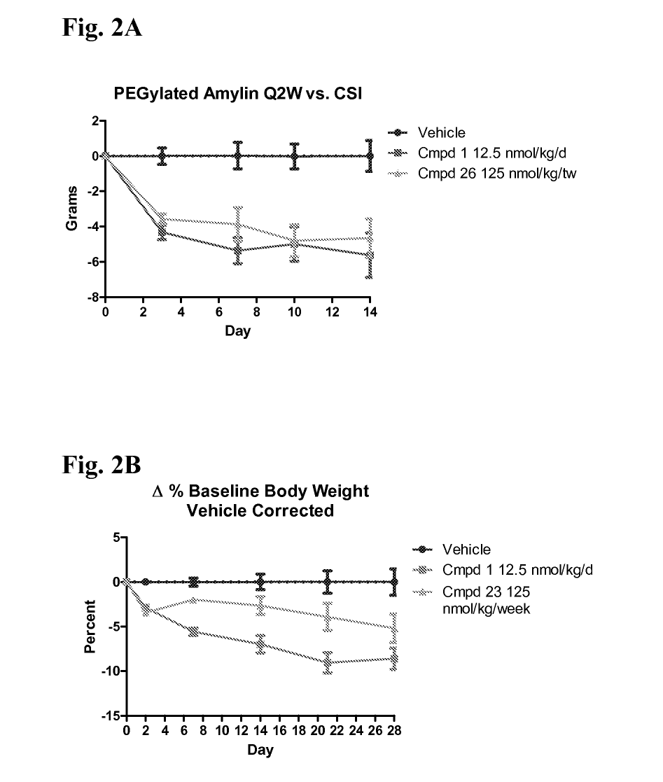

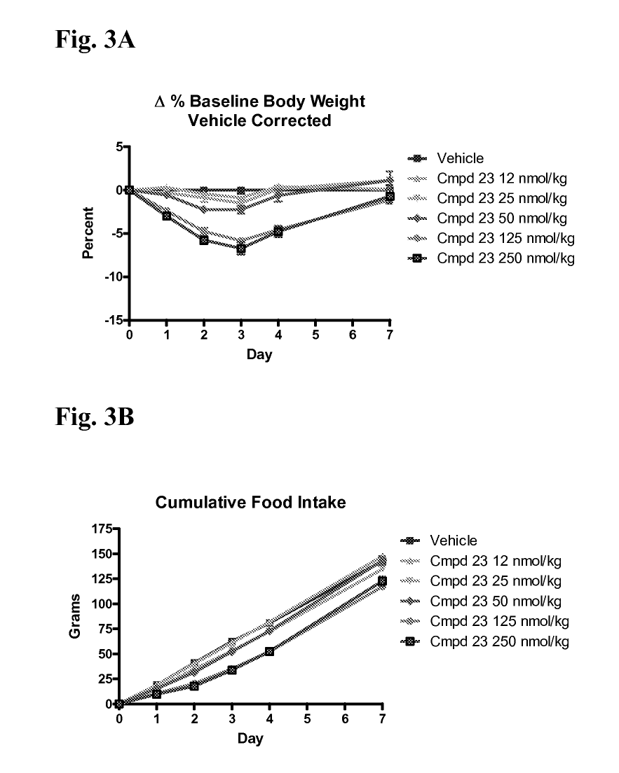

The effect on weight loss, as judged with SC injection, of a twice a week dose or once a week dose of Cmpd 26, was investigated. When dosed twice a week at 125 nmol/kg in a DIO (“diet-induced obese”) rat, Cmpd 26 has similar efficacy as a continuous infusion of 12.5 nmol/kg/d Cmpd 1 (FIG. 2A). Cmpd 23 dosed once a week at 125 nmol/kg was not as efficacious as infused Cmpd 1 when given to DIO rats for four weeks, but did show consistent lowering of body weight (FIG. 2B). Cmpd 23 also reduced body weight and food intake in a dose dependent fashion in lean rats, as shown in FIGS. 3A-3B.

### Example 6

### Effect of Pegylation on Food Intake: Cmpds 19, 23, 27

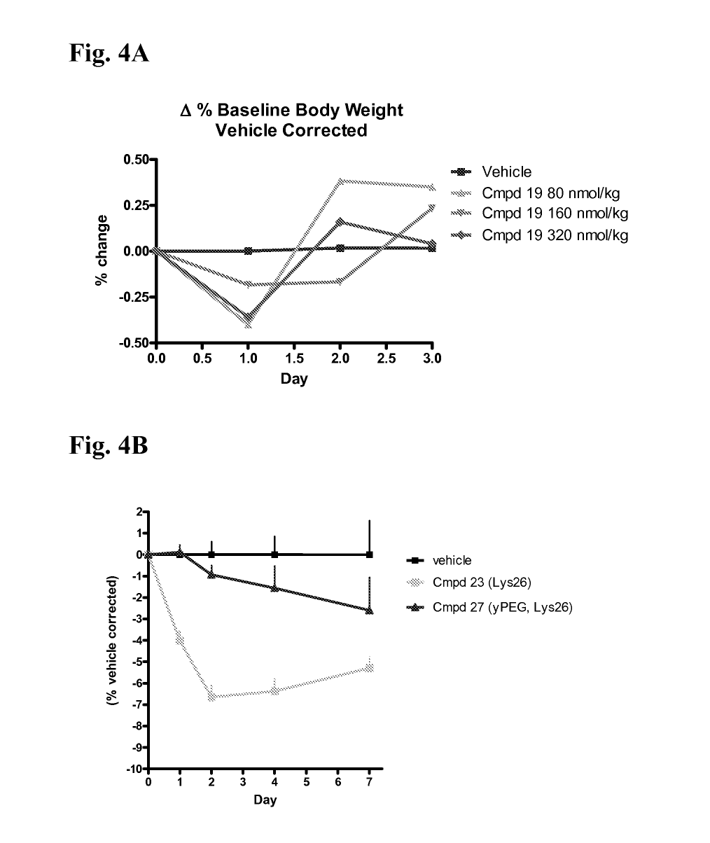

The effect on 24-hour food intake, as judged with SC injection, of a single dose of a compound having either a y-branched PEG (Cmpd 27) or an N-terminal PEG (Cmpd 19), was investigated. As shown in FIG. 4A, three doses of the N-terminal pegylated compound, Cmpd 19, were not as efficacious as the vehicle in reducing body weight in DIO rats. The y-branched pegylated compound, Cmpd 27, was not as efficiacious as the linear pegylated version, Cmpd 23, in reducing body weight in lean rats, as shown in FIG. 4B.

### Example 7

### Effect of Pegylation on Food Intake: Cmpds 26, 28, 29, 30, 31

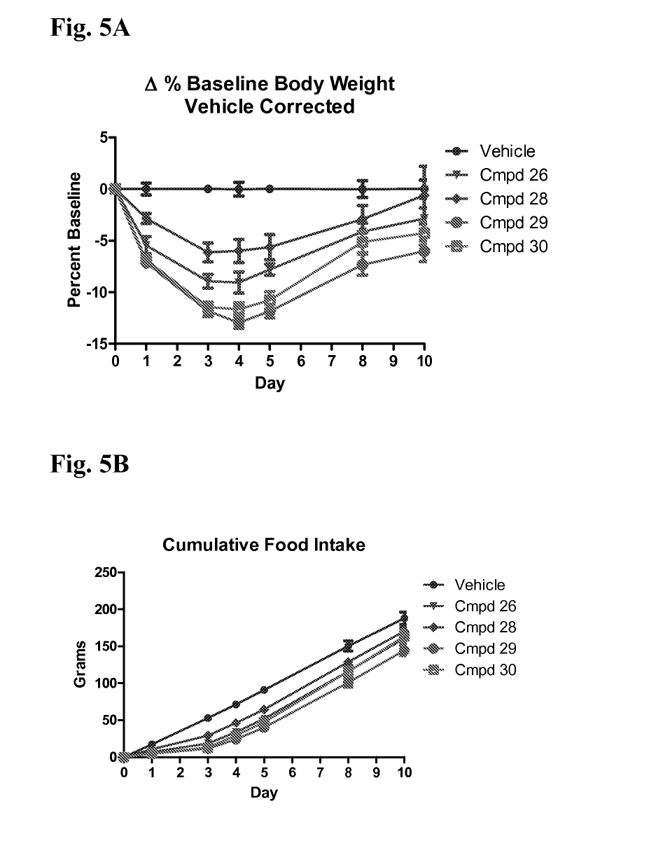

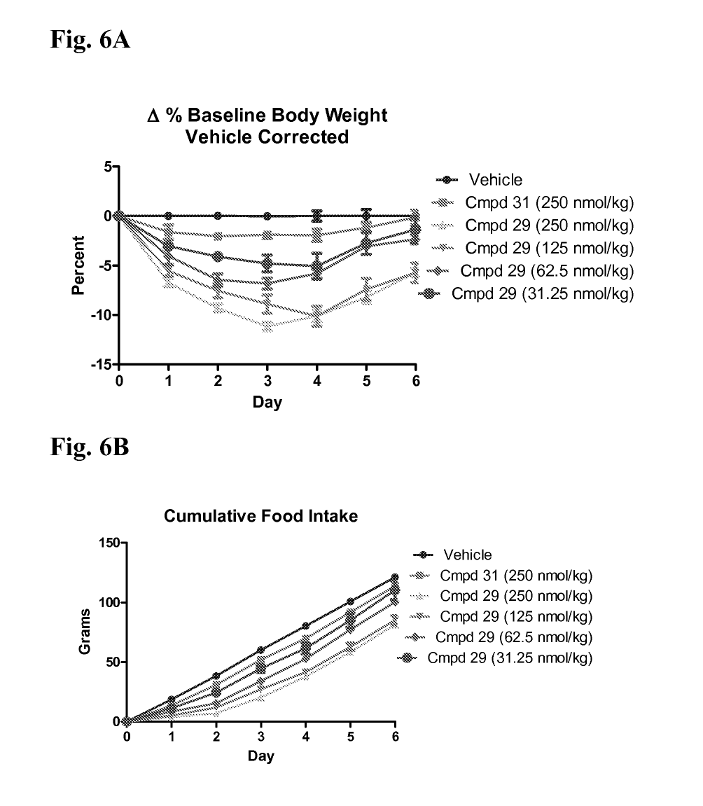

The effect on 24-hour food intake, as judged with SC injection, was investigated for Cmpds 26, 28, 29 and 30. As shown in FIG. 5A-5B, each of the tested pegylated compounds 28, 29, and 30 were at least as efficacious as Cmpd 26 in body weight and food intake reduction in lean rats. The y-branched pegylated compound, Cmpd 31, was not as efficiacious as the linear pegylated version, Cmpd 29, in body weight and food intake reduction in lean rats, as shown in FIG. 6A-6B. Cmpd 29 also showed dose dependent efficacy, as demonstrated in FIG. 6A-6B.

### Example 8

### Effect of Pegylation on Food Intake: Cmpds 29, 32, 33, 34, 35 and 36

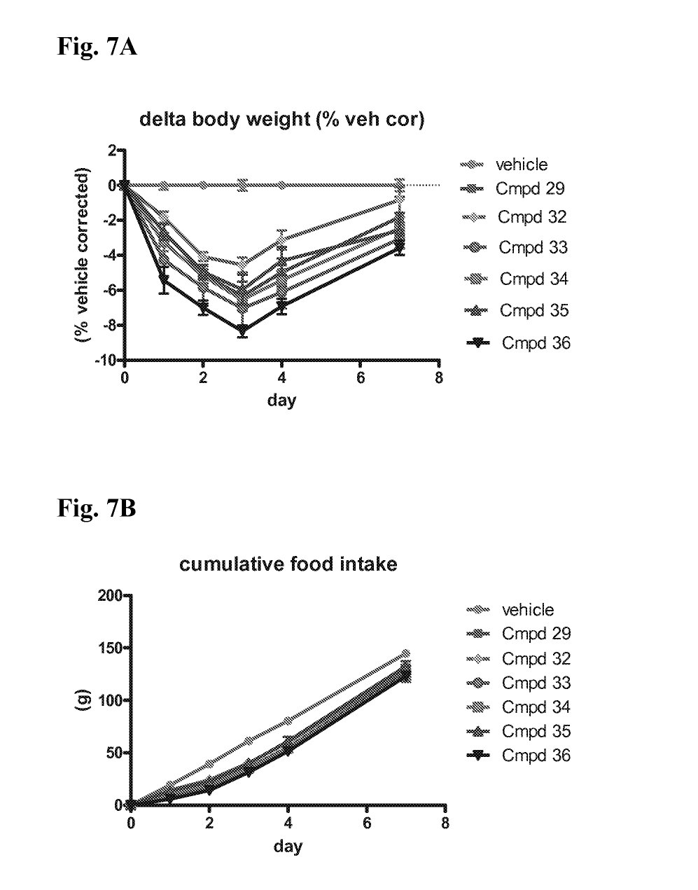

The effect on cumulative food intake and body weight reduction, as judged with SC injection, was investigated for Cmpds 29, 32, 33, 34, 35 and 36. As shown in FIG. 7A-7B, each of the tested pegylated compounds was efficacious in body weight and food intake reduction in lean rats at 125 nmol/kg. In the case of most of the compounds, weight loss was still evident even after one week.

### Example 9

### Effect of Pegylation on Food Intake: Cmpds 41 and 42

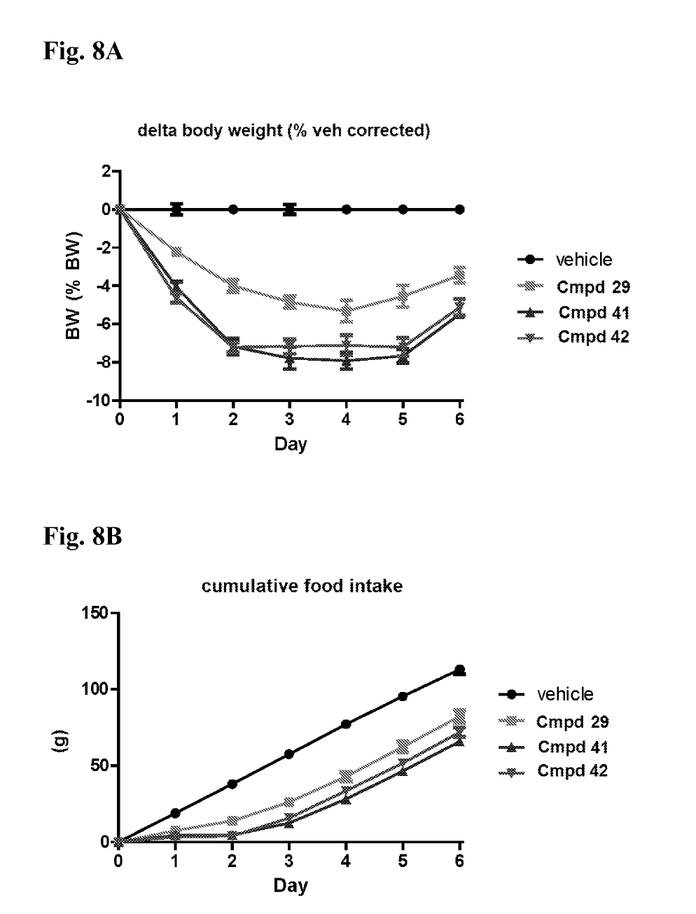

The effect on cumulative food intake and body weight reduction, as judged with SC injection, was investigated for Cmpds 41 and 42. As shown in FIG. 8A-8B, each of the tested pegylated compounds was efficacious in body weight and food intake reduction in lean rats at 125 nmol/kg.

In summary, the food intake data set forth in Examples 4-9 provides valuable observations regarding the efficacy and effect on duration of action of pegylation of the polypeptide element of the tested compounds. Specifically, 40 KD PEG derivatives of polypeptide components exhibit an extended time course of action compared to the non-pegylated peptide. The attachment of the PEG at positions 21, 26, or 31 increased both duration of action and the magnitude of the food intake response. Linear PEG compounds demonstrate greater efficacy in the food intake assay compared to the branched PEG compounds.

What is claimed is:

1. A polypeptide conjugate comprising a polypeptide component covalently linked to a duration enhancing moiety, wherein the polypeptide component comprises an amino acid sequence of residues 1-37 of Formula (I):
(I)
(SEQ ID NO: 4)
X′-Xaa¹-Cys²-Asn³-Thr⁴-Ala⁵-Thr⁶-Cys⁷-Ala⁸-Thr⁹-
Gln¹⁰-Arg¹¹-Leu¹²-Ala¹³-Asn¹⁴-Phe¹⁵-Leu¹⁶-Val¹⁷-Xaa¹⁸-
Ser¹⁹-Ser²⁰-Xaa²¹-Asn²²-Phe²³-Xaa²⁴-Xaa²⁵-Xaa²⁶-Xaa²⁷-
Xaa²⁸-Xaa²⁹-Thr³⁰-Xaa³¹-Val³²-Gly³³-Xaa³⁴-Xaa³⁵-Thr³⁶-
Tyr³⁷-X
wherein up to 25% of the amino acids set forth in Formula (I) may be deleted or substituted with a different amino acid; wherein
X′ is hydrogen, an N-terminal capping group, a bond to a duration enhancing moiety, or a linker to a duration enhancing moiety;
Xaa¹ is Lys or a bond;
Xaa¹⁸ is Lys, Cys, or His,
Xaa²¹ is Lys, Cys, or Asn;
Xaa²⁴ is Lys, Cys, or Gly;
Xaa²⁵ is Lys, Cys, or Pro;
Xaa²⁶ is Lys, Cys, or Ile;
Xaa²⁷ is Lys, Cys, or Leu;
Xaa²⁸ is Lys, Cys, or Pro;
Xaa²⁹ is Lys, Cys, or Pro;
Xaa³¹ is Lys, Cys, or Asn;
Xaa³⁴ is Lys, Cys, or Ser; and
Xaa³⁵ is Lys, Cys, or Asn,
X is substituted or unsubstituted amino, substituted or unsubstituted alkylamino, substituted or unsubstituted dialkylamino, substituted or unsubstituted cycloalkylamino, substituted or unsubstituted arylamino, substituted or unsubstituted aralkylamino, substituted or unsubstituted alkyloxy, substituted or unsubstituted aryloxy, substituted or unsubstituted aralkyloxy, hydroxyl, a bond to a duration enhancing moiety, or a linker to a duration enhancing moiety; wherein the duration enhancing moiety is covalently linked, optionally through a linker, to a side chain of a linking amino acid residue, X′ or X.

2. The polypeptide conjugate according to claim 1, wherein the duration enhancing moiety is a polyethylene glycol or a derivative thereof.

3. The polypeptide conjugate of claim 1, wherein the linking amino acid residue is cysteine or lysine.

4. The polypeptide conjugate of claim 1, wherein the polyethylene glycol is linear, branched or comb type.

5. The polypeptide conjugate of claim 1, wherein the polypeptide conjugate comprises one duration enhancing moiety.

6. The polypeptide conjugate of claim 1, wherein the duration enhancing moiety is attached to the N-terminal amino acid residue of the polypeptide.

7. The polypeptide conjugate of claim 1, wherein the duration enhancing moiety is attached to the C-terminal amino acid residue of the polypeptide.

8. The polypeptide conjugate of claim 1, wherein the duration enhancing moiety is attached to the side chain of the amino acid at position 11, 18, 24-29, 31, 34, or 35.

9. A pharmaceutical composition comprising a polypeptide conjugate according to claim 1, and a pharmaceutically acceptable excipient.

10. A method for treating a disease or disorder in a subject comprising administering a composition according to claim 9 to a subject in need of treatment in an amount effective to treat the disease or disorder.

11. The method according to claim 10, wherein the disease or disorder is an eating disorder, insulin resistance, obesity, overweight, abnormal postprandial hyperglycemia, Type I diabetes, Type II diabetes, gestational diabetes, metabolic syndrome, dumping syndrome, hypertension, dyslipidemia, cardiovascular disease, hyperlipidemia, sleep apnea, cancer, pulmonary hypertension, cholescystitis, osteoarthritis, or short bowel syndrome.


SEQUENCE LISTING (57 sequences)

SEQ ID NO: 1 - protein, 37 aa, Rattus sp.
KCNTATCATQRLANFLVRSSNNLGPVLPPTNVGSNTY

SEQ ID NO: 2 - protein, 37 aa, Homo sapiens [sequence omitted]

SEQ ID NO: 3 - protein, 37 aa, Artificial Sequence
  Description of Artificial Sequence Synthetic polypeptide
KCNTATCATQRLANFLVHSSNNFGPILPPTNVGSNTY

SEQ ID NO: 4 - protein, 37 aa, Artificial Sequence
  Description of Artificial Sequence Synthetic polypeptide
KCNTATCATQRLANFLVXSSXNFXXXXXXTXVGXXTY

SEQ ID NO: 5 - protein, 4 aa, Artificial Sequence
  Description of Artificial Sequence Synthetic peptide
GGGG

SEQ ID NO: 6 - protein, 5 aa, Artificial Sequence
  Description of Artificial Sequence Synthetic peptide
GGGGG

SEQ ID NO: 7 - protein, 8 aa, Artificial Sequence0123WO1
  Description of Artificial Sequence Synthetic peptide
GGGKGGGG

SEQ ID NO: 8 - protein, 8 aa, Artificial Sequence
  Description of Artificial Sequence Synthetic peptide
GGGNGSGG

SEQ ID NO: 9 - protein, 8 aa, Artificial Sequence
  Description of Artificial Sequence Synthetic peptide
GGGCGGGG

SEQ ID NO: 10 - protein, 5 aa, Artificial Sequence
  Description of Artificial Sequence Synthetic peptide
GPNGG

SEQ ID NO: 11 - protein, 12 aa, Artificial Sequence
  Description of Artificial Sequence Synthetic peptide
GSGSGSGSGSGS

SEQ ID NO: 12 - protein, 18 aa, Artificial Sequence
  Description of Artificial Sequence Synthetic peptide
GGSGGSGGSGGSGGSGGS

SEQ ID NO: 13 - protein, 24 aa, Artificial Sequence
  Description of Artificial Sequence Synthetic peptide
GGGSGGGSGGGSGGGSGGGSGGGS

SEQ ID NO: 14 - protein, 30 aa, Artificial Sequence
  Description of Artificial Sequence Synthetic polypeptide
GGGGSGGGGSGGGGSGGGGSGGGGSGGGGS

SEQ ID NO: 15 - protein, 15 aa, Artificial Sequence
  Description of Artificial Sequence Synthetic peptide
GGGGSGGGGSGGGGS

SEQ ID NO: 16 - protein, 37 aa, Artificial Sequence; identical to SEQ ID NO: 1
  Description of Artificial Sequence Synthetic polypeptide

SEQ ID NO: 17 - protein, 36 aa, Artificial Sequence
  Description of Artificial Sequence Synthetic polypeptide
CNTATCATQRLANFLVRSSNNLGPVLPPTNVGSNTY

SEQ ID NO: 18 - protein, 37 aa, Artificial Sequence
  Description of Artificial Sequence Synthetic polypeptide
KCNTATCATQRLANFLVRSSKNLGPVLPPTNVGSNTY

SEQ ID NO: 19 - protein, 36 aa, Artificial Sequence
  Description of Artificial Sequence Synthetic polypeptide
CNTATCATQRLANFLVRSSKNLGPVLPPTNVGSNTY

SEQ ID NO: 20 - protein, 37 aa, Artificial Sequence
  Description of Artificial Sequence Synthetic polypeptide
KCNTATCATQRLANFLVRSSNNLGPKLPPTNVGSNTY

SEQ ID NO: 21 - protein, 36 aa, Artificial Sequence
  Description of Artificial Sequence Synthetic polypeptide
CNTATCATQRLANFLVRSSNNLGPKLPPTNVGSNTY

SEQ ID NO: 22 - protein, 37 aa, Artificial Sequence
  Description of Artificial Sequence Synthetic polypeptide
KCNTATCATQRLANFLVRSSNNLGPVLPPTKVGSNTY

SEQ ID NO: 23 - protein, 36 aa, Artificial Sequence
  Description of Artificial Sequence Synthetic polypeptide
CNTATCATQRLANFLVRSSNNLGPVLPPTKVGSNTY

SEQ ID NO: 24 - protein, 37 aa, Artificial Sequence; identical to SEQ ID NO: 3
  Description of Artificial Sequence Synthetic polypeptide

SEQ ID NO: 25 - protein, 36 aa, Artificial Sequence
  Description of Artificial Sequence Synthetic polypeptide
CNTATCATQRLANFLVHSSNNFGPILPPTNVGSNTY

SEQ ID NO: 26 - protein, 36 aa, Artificial Sequence
  Description of Artificial Sequence Synthetic polypeptide
CNTATCATQRLANFLVHSSKNFGPILPPTNVGSNTY

SEQ ID NO: 27 - protein, 36 aa, Artificial Sequence
  Description of Artificial Sequence Synthetic polypeptide
CNTATCATQRLANFLVHSSNNFGPKLPPTNVGSNTY

SEQ ID NO: 28 - protein, 36 aa, Artificial Sequence
  Description of Artificial Sequence Synthetic polypeptide
CNTATCATQRLANFLVHSSNNFGPILPPTKVGSNTY

SEQ ID NO: 29 - protein, 36 aa, Artificial Sequence
  Description of Artificial Sequence Synthetic polypeptide
CNTATCATQRLANFLVHSSNNFKPILPPTNVGSNTY

SEQ ID NO: 30 - protein, 36 aa, Artificial Sequence
  Description of Artificial Sequence Synthetic polypeptide
CNTATCATQRLANFLVHSSNNFGKILPPTNVGSNTY

SEQ ID NO: 31 - protein, 36 aa, Artificial Sequence
  Description of Artificial Sequence Synthetic polypeptide
CNTATCATQRLANFLVHSSNNFGPIKPPTNVGSNTY

SEQ ID NO: 32 - protein, 36 aa, Artificial Sequence
  Description of Artificial Sequence Synthetic polypeptide
CNTATCATQRLANFLVHSSNNFGPILKPTNVGSNTY

SEQ ID NO: 33 - protein, 36 aa, Artificial Sequence
  Description of Artificial Sequence Synthetic polypeptide
CNTATCATQRLANFLVHSSNNFGPILPKTNVGSNTY

SEQ ID NO: 34 - protein, 36 aa, Artificial Sequence
  Description of Artificial Sequence Synthetic polypeptide
CNTATCATQRLANFLVKSSNNFGPILPPTNVGSNTY

SEQ ID NO: 35 - protein, 36 aa, Artificial Sequence
  Description of Artificial Sequence Synthetic polypeptide
CNTATCATQRLANFLVHSSNNFGPILPPTNVGKNTY

SEQ ID NO: 36 - protein, 36 aa, Artificial Sequence
  Description of Artificial Sequence Synthetic polypeptide
CNTATCATQRLANFLVHSSNNFGPILPPTNVGSKTY

SEQ ID NO: 37 - protein, 37 aa, Artificial Sequence; identical to SEQ ID NO: 1
  Description of Artificial Sequence Synthetic polypeptide

SEQ ID NO: 38 - protein, 36 aa, Artificial Sequence; identical to SEQ ID NO: 17
  Description of Artificial Sequence Synthetic polypeptide

SEQ ID NO: 39 - protein, 37 aa, Artificial Sequence; identical to SEQ ID NO: 18
  Description of Artificial Sequence Synthetic polypeptide

SEQ ID NO: 40 - protein, 36 aa, Artificial Sequence; identical to SEQ ID NO: 19
  Description of Artificial Sequence Synthetic polypeptide

SEQ ID NO: 41 - protein, 37 aa, Artificial Sequence; identical to SEQ ID NO: 20
  Description of Artificial Sequence Synthetic polypeptide

SEQ ID NO: 42 - protein, 36 aa, Artificial Sequence; identical to SEQ ID NO: 21
  Description of Artificial Sequence Synthetic polypeptide

SEQ ID NO: 43 - protein, 37 aa, Artificial Sequence; identical to SEQ ID NO: 22
  Description of Artificial Sequence Synthetic polypeptide

SEQ ID NO: 44 - protein, 36 aa, Artificial Sequence; identical to SEQ ID NO: 23
  Description of Artificial Sequence Synthetic polypeptide

SEQ ID NO: 45 - protein, 37 aa, Artificial Sequence; identical to SEQ ID NO: 20
  Description of Artificial Sequence Synthetic polypeptide

SEQ ID NO: 46 - protein, 36 aa, Artificial Sequence; identical to SEQ ID NO: 26
  Description of Artificial Sequence Synthetic polypeptide

SEQ ID NO: 47 - protein, 36 aa, Artificial Sequence; identical to SEQ ID NO: 27
  Description of Artificial Sequence Synthetic polypeptide

SEQ ID NO: 48 - protein, 36 aa, Artificial Sequence; identical to SEQ ID NO: 28
  Description of Artificial Sequence Synthetic polypeptide

SEQ ID NO: 49 - protein, 36 aa, Artificial Sequence; identical to SEQ ID NO: 27
  Description of Artificial Sequence Synthetic polypeptide

SEQ ID NO: 50 - protein, 36 aa, Artificial Sequence; identical to SEQ ID NO: 29
  Description of Artificial Sequence Synthetic polypeptide

SEQ ID NO: 51 - protein, 36 aa, Artificial Sequence; identical to SEQ ID NO: 30
  Description of Artificial Sequence Synthetic polypeptide

SEQ ID NO: 52 - protein, 36 aa, Artificial Sequence; identical to SEQ ID NO: 31
  Description of Artificial Sequence Synthetic polypeptide

SEQ ID NO: 53 - protein, 36 aa, Artificial Sequence; identical to SEQ ID NO: 32
  Description of Artificial Sequence Synthetic polypeptide

SEQ ID NO: 54 - protein, 36 aa, Artificial Sequence; identical to SEQ ID NO: 33
  Description of Artificial Sequence Synthetic polypeptide

SEQ ID NO: 55 - protein, 36 aa, Artificial Sequence; identical to SEQ ID NO: 34
  Description of Artificial Sequence Synthetic polypeptide

SEQ ID NO: 56 - protein, 36 aa, Artificial Sequence; identical to SEQ ID NO: 35
  Description of Artificial Sequence Synthetic polypeptide

SEQ ID NO: 57 - protein, 36 aa, Artificial Sequence; identical to SEQ ID NO: 36
  Description of Artificial Sequence Synthetic polypeptide